(19) United States
(12) Patent Application Publication
(10) Pub. No.: US 2024/0041375 A1
(43) Pub. Date: Feb. 8, 2024

(54) WETTING AND FRACTURE INDUCED COMPOSITES FOR HIGHLY SENSITIVE RESISTIVE AND CAPACITIVE SENSORS

(71) Applicant: University of Washington, Seattle, WA (US)

(72) Inventors: Jae-Hyun Chung, Seattle, WA (US); Jinyuan G. Zhang, Seattle, WA (US); Anthony B. Dichiara, Seattle, WA (US); Zhongjie Qian, Seattle, WA (US)

(73) Assignee: University of Washington, Seattle, WA (US)

(21) Appl. No.: 18/258,776

(22) PCT Filed: Dec. 20, 2021
(86) PCT No.: PCT/US2021/064410

§ 371 Date: Jun. 21, 2023

Related U.S. Application Data
(60) Provisional application No. 63/130,141, filed on Dec. 23, 2020.

(51) Int. Cl. A61B 5/263; D21H 17/67; D21H 21/14; D21H 27/00; D21H 19/68; D06M 11/74; D06M 23/16; D06M 11/05; G01L 1/18; G01L 1/14

(52) U.S. Cl. CPC A61B 5/263; D21H 17/67; D21H 21/14; D21H 27/00; D21H 19/68; D06M 11/74; D06M 23/16; D06M 11/05; G01L 1/18; G01L 1/142; A61B 2562/029; A61B 2562/12; D06M 2101/06

(57) ABSTRACT

A sensor, comprising including a composite substrate including a template material, where the template material includes a plurality of insulating fibers, and a plurality of carbon nanotubes bonded to the insulating fibers forming a nanotube coating on the insulating fibers, and where the composite substrate exhibits a tensional fracture induced by a unidirectional tensile force to the composite substrate, wherein the plurality of insulating fibers align along the tensile force and expand in an out-of-plane direction at the site of the fracture, a first electrode coupled to the nanotube coating on one side of the fracture, and a second electrode coupled to the nanotube coating on the opposite side of the fracture, such that an electrical signal applied between the first electrode and the second electrode passes through the plurality of junctions at the site of the fracture.

### CROSS-REFERENCE TO RELATED APPLICATION

This application claims the benefit of U.S. Provisional Application 63/130141, filed on Dec. 23, 2020, which is hereby incorporated in its entirety.

### BACKGROUND

Auxetic materials, characterized by their negative Poisson's ratios, expand in the transverse direction under uniaxial stretching. This distinctive trait offers unique mechanical properties, namely indentation resistance, fracture toughness, and shear resistance, which makes auxetic materials appealing in diverse fields, such as tissue engineering, aerospace, and sports. Auxetic materials showing a negative Poisson's ratio can offer unique sensing capability due to drastic percolation change.

However, the manufacturing of periodically arranged structures for practical applications remains challenging, and random structures are typically associated with only modest Poisson's ratios. Additionally, while auxetic-based resistive sensors have been developed for various applications ranging from healthcare, to human-machine interfaces, and automations, few reports on capacitive sensing of auxetic materials has been presented.

Accordingly, there exists a need for fabricated auxetic capacitive sensors that can be used in various wearable applications, which can also be conceived at low cost. There also exists a need for methods of manufacturing of auxetic materials in a controlled manner.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Described herein is a novel way to control the fracture of carbon nanotube (CNT) paper composites (CPC) with a great spatial resolution based on a scalable liquid (e.g., water)-printing method to enhance the auxetic behavior of fibrous composites for highly sensitive piezo-resistivity. A noncontact printing of water can locally weaken the hydrogen bonds and soften the pulp fibers for controlled fractures. Further, disclosed is the effect of the wetting process on the piezoresistive sensitivity of said fibers.

The produced CPC piezoresistive sensors are characterized for sensitivity, dynamic range, and reproducibility and are applied to multiple wearable devices, such as pulse detection, breath monitoring and walk pattern recognition. The acquired auxetic behavior from the random network structures opens the way to develop high-performance and low-cost sensors for a large variety of applications in portable electronics.

In one aspect, a sensor, comprising a composite substrate comprising a template material, wherein the template material comprises a plurality of insulating fibers, and a plurality of carbon nanotubes bonded to the insulating fibers forming a nanotube coating on the insulating fibers, wherein the composite substrate exhibits a tensional fracture induced by a unidirectional tensile force to the composite substrate, wherein the plurality of insulating fibers align along the tensile force and expand in an out-of-plane direction at the site of the fracture, a first electrode coupled to the nanotube coating on one side of the fracture; and a second electrode coupled to the nanotube coating on the opposite side of the fracture, such that an electrical signal applied between the first electrode and the second electrode passes through the plurality of junctions at the site of the fracture is disclosed.

In another aspect, a method of making a sensor comprising applying a unidirectional tensile force to the composite substrate, wherein the plurality of insulating fibers aligns along the tensile force and bulging with out-of-plane direction at the site of a tensional fracture, wherein the precursor composite substrate comprises a composite substrate comprising a template material, wherein the template material comprises a plurality of insulating fibers, and a plurality of carbon nanotubes bonded to the insulating fibers forming a nanotube coating on the insulating fibers, and a first electrode coupled to the nanotube coating on one side of the fracture and a second electrode coupled to the nanotube coating on the opposite side of the fracture, such that an electrical signal applied between the first electrode and the second electrode passes through the plurality of junctions at the site of the fracture is disclosed.

In another aspect, a sensor manufactured by any of the methods described herein is disclosed.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a system for water printing under uniaxial tension to produce auxetic CPCs, in accordance with the present technology;

FIG. 2A is a graph showing the stress—strain relationship coupled with normalized resistance change, in accordance with the present technology;

FIG. 2B is a graph showing Instantaneous Poisson's ratios of pure paper and CPC with CNT wt % of 2.5, 5 and 10 during stretching, in accordance with the present technology;

FIG. 2C shows simulation result of stress distribution for CPC under tension, in accordance with the present technology;

FIG. 2D shows is a graph of the maximum effective Poisson's ratios for water-printed and non- pure paper and CPC with CNT wt % of 2.5, 5, and 10, in accordance with the present technology;

FIG. 3A is a SEM image and fiber orientation of 2.5 CNT wt %-CPC at strain of 0, in accordance with the present technology;

FIG. 3B is a SEM image and fiber orientation of 2.5 CNT wt %-CPC at strain of 0.03, in accordance with the present technology;

FIG. 3C is a SEM images and fiber orientation of 2.5 CNT wt %-CPC at strain of and 0.10, in accordance with the present technology;

FIG. 3D is SEM image of fractured CPC with 10 CNT wt % at strain of 0.10 in accordance with the present technology;

FIG. 3E is a SEM image of a pristine CPC, in accordance with the present technology;

FIG. 3F shows cellulose fibers buckled, and forcing each other into the out of plane direction, in accordance with the present technology;

FIGS. 4A-4F are graphs showing characterizations of the sensing performance of a CPC piezoresistive sensor, in accordance with the present technology;

FIG. 5A is a CPC piezoelectric heartbeat sensor capable of measuring the rate of cardiovascular pulsations when wrapped around the wrist of an individual, in accordance with the present technology;

FIG. 5B depicts a CPC piezoelectric sensor on a belt, in accordance with the present technology;

FIG. 5C shows the resistance changes of a foot pressure sensor at three modes of motions, in accordance with the present technology;

FIGS. 6A-6C are graphs of the stress-strain relationship between 2.5%, 5%, and 10% CNT after no wetting, 2, 6, and 10 times of wetting in accordance with the present technology;

FIGS. 6D-6F are graphs of the wetting time in relation to the fracture strain of the CNT, the ultimate strength in MPa, and the wet strength retention, in accordance with the present technology;

FIG. 7A is a test setup to investigate the auxetic behavior of the CPC, in accordance with the present technology;

FIG. 7B is a fractured CPC with and without water printing, in accordance with the present technology;

FIG. 7C is a graph of the stress-strain relationship for the CPC with and without water printing, in accordance with the present technology;

FIG. 7D is the capacitance change for CPC with and without water printing, in accordance with the present technology;

FIG. 8A-8C are SEM images at 0.12, 0.15, and 0.18 strain, in accordance with the present technology;

FIG. 8D is a graph of the normalized thickness change according to axial strain for CPC with and without water printing, in accordance with the present technology;

FIG. 8E is a graph showing the Poisson's ratio according to specimen widths, in accordance with the present technology;

FIG. 8F is a graph showing the maximum capacitance according to sample widths;

FIG. 9A is the stress distribution on a 1 mm width CPC strip resulting from the compression, in accordance with the present technology;

FIG. 9B is the stress distribution on a 3 mm width CPC strip, in accordance with the present technology;

FIG. 9C is the compressive stress built across the width, in accordance with the present technology;

FIG. 9D is a graph showing that at 1 mm width, the averaged engineering stress cannot buckle the central region, in accordance with the present technology;

FIGS. 10A-10F are graphs showing the resistance and capacitance change of the specimen of 0.10, 0.12, 0.15, 0.18 and 0.24-strain for the humidity change, in accordance with the present technology;

FIG. 11A is a graph of the capacitance change of a fractured CPC sensor according to humidity change, in accordance with the present technology;

FIG. 11B is a graph of the comparison of the fractured CPC-humidity response to a commercial sensor;

FIGS. 12A-12D are graphs of the capacitive changes of PAA-coated CPC, trimmed-CPC, plastic-film-coated CPC, and trimmed aluminum sensors for cyclic humidity change, in accordance with the present technology;

FIG. 13A shows a chamber to measure humidity change on a hand, in accordance with the present technology; and

FIG. 13B is a graph of the capacitance change measured on palm, in accordance with the present technology.

### DETAILED DESCRIPTION

While illustrative embodiments have been illustrated and described, it will be appreciated that various changes can be made therein without departing from the spirit and scope of the invention.

Generally, the technology described below is a capacitive sensor comprising carbon nanotubes deposed about paper fiber. Further preparation of the composite material for capacitive sensing occurs when paper fibers and carbon nanotubes are aligned via a tensile fracture of the composite sensor material.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

In one aspect, a sensor, comprising a composite substrate comprising a template material, wherein the template material comprises a plurality of insulating fibers, and a plurality of carbon nanotubes bonded to the insulating fibers forming a nanotube coating on the insulating fibers, wherein the composite substrate exhibits a tensional fracture induced by a unidirectional tensile force to the composite substrate, wherein the plurality of insulating fibers align along the tensile force and expand in an out-of-plane direction at the site of the fracture, a first electrode coupled to the nanotube coating on one side of the fracture; and a second electrode coupled to the nanotube coating on the opposite side of the fracture, such that an electrical signal applied between the first electrode and the second electrode passes through the plurality of junctions at the site of the fracture is disclosed.

The composite substrate may be carbon nanotube (CNT) paper composites (CPC). In some embodiments, the template material is a paper composite containing insulating fibers. In the composite, CNTs offer electrical conductivity while cellulose fibers offer the structural frame. Since cellulose fibers are the structural component of a composite, the deformation of cellulose fibers contributes to the auxetic behavior under stretching.

The auxetic behavior of CPC has been characterized for elastic and plastic regions. The auxetic materials exhibiting negative Poisson's ratio are frequently observed in fibrous materials. Paper and non-woven fabrics possess the auxetic behavior. Periodic, repeating structures were designed to demonstrate auxeticity.

In some embodiments, the composite substrate is formed. In some embodiments, CNT composite papers are formed. In some embodiments, the CPC are formed with a hand-sheet molder. In some embodiments, prior to formation, CNT-OH is dispersed and added to the pulp mixture of the composite papers in order to achieve a uniform distribution of charge transport routes throughout the final composition. In some embodiments, the composite papers have a total mass of 1.2 g OD. In some embodiments, the density of the CPC is between 50-100 g/m², which was optimum. In some embodiments the CPC have 2.5, 5, or 10 wt % of CNT. In some embodiments, the width of the CPC ranges from 1-10 mm. In some embodiments, the width of the CPC is 1 mm, 3 mm, 5 mm, 7 mm, or 10 mm.

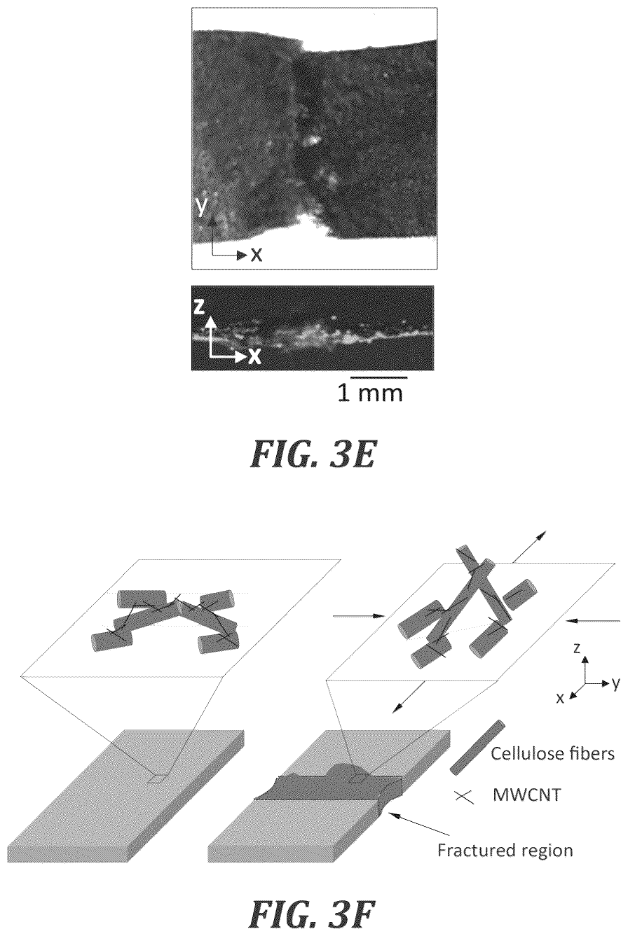

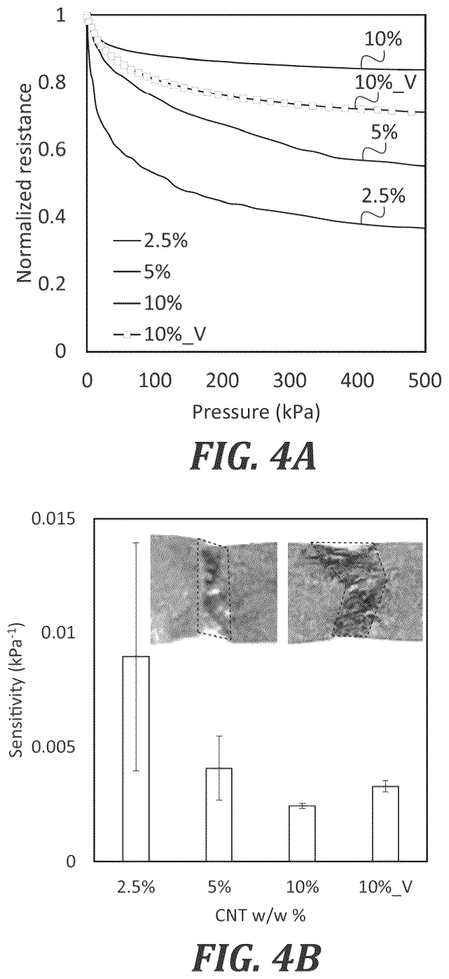

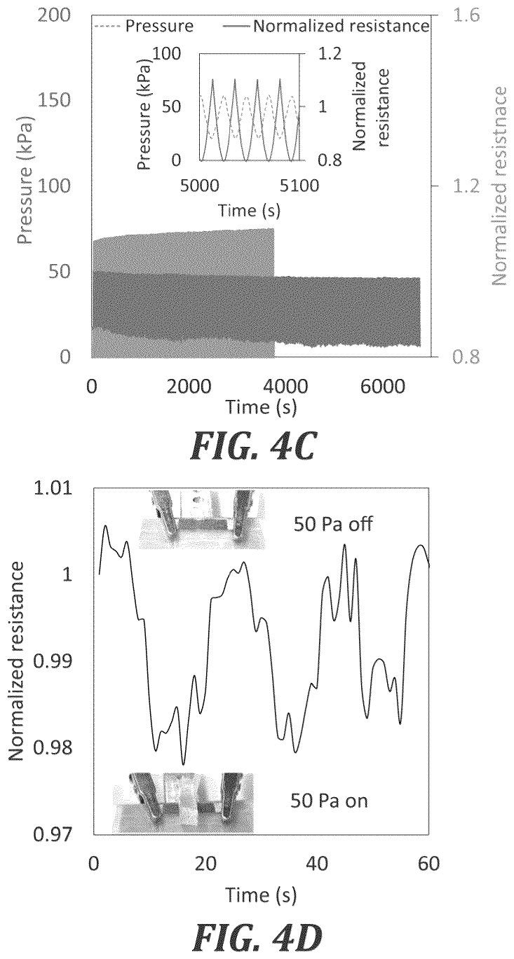

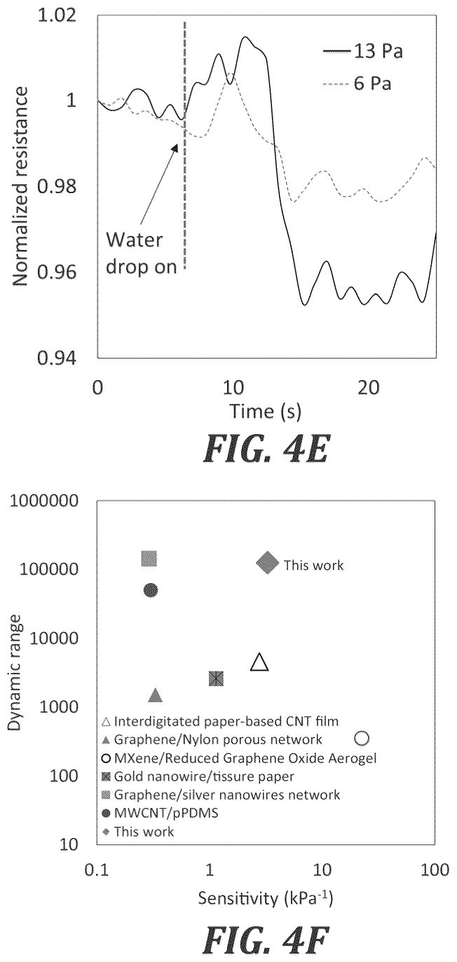

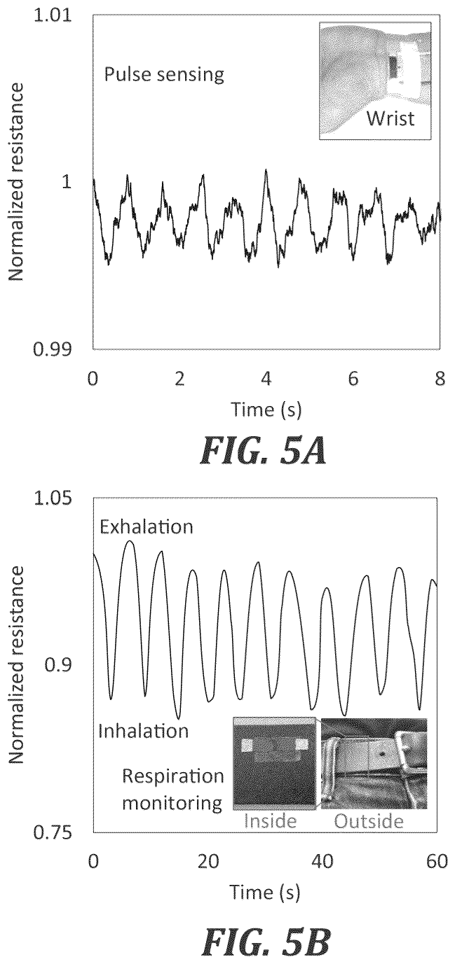

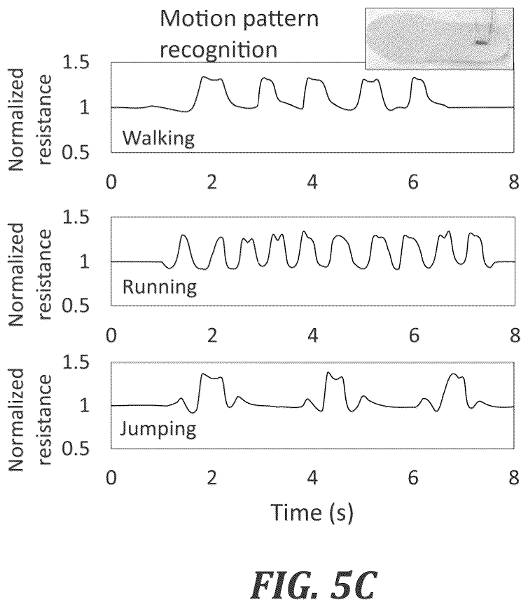

The CPC is stretched to form a fracture. In some embodiments, the fracture is propagated at a 45-degree angle to the stretching direction. An example sensor made from CPC and stretched to form a fracture is illustrated in FIG. 3F. When the CPC is stretched, a fractured region is formed. In this fractured region, the CNT coated insulating fibers are buckled out-of-plane, as described below.

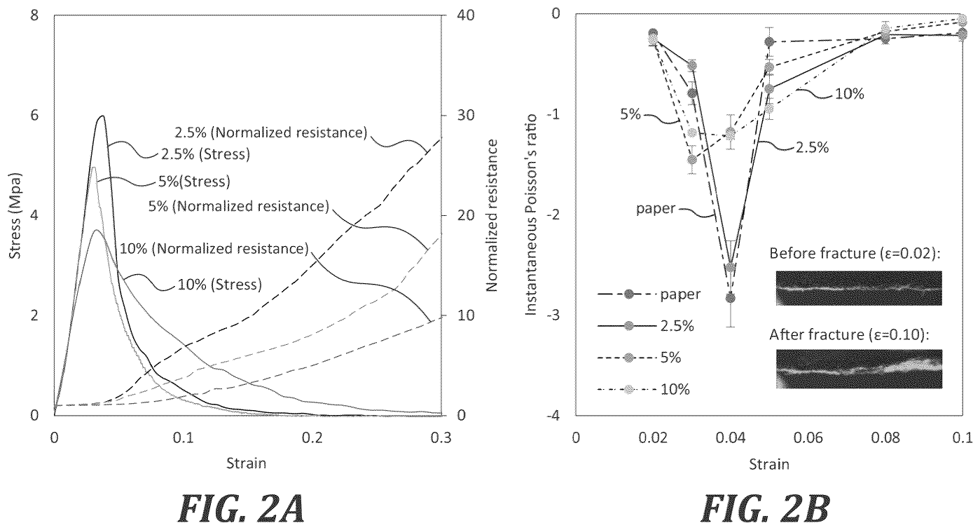

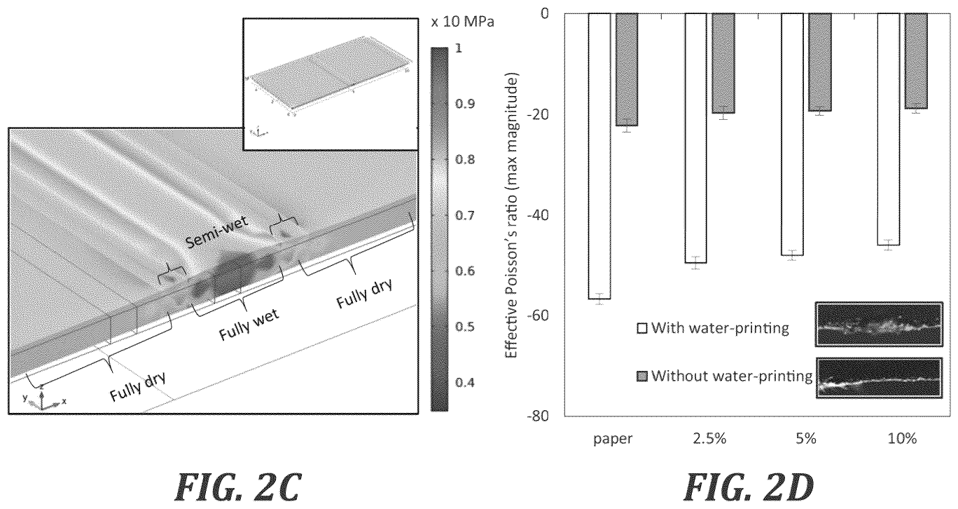

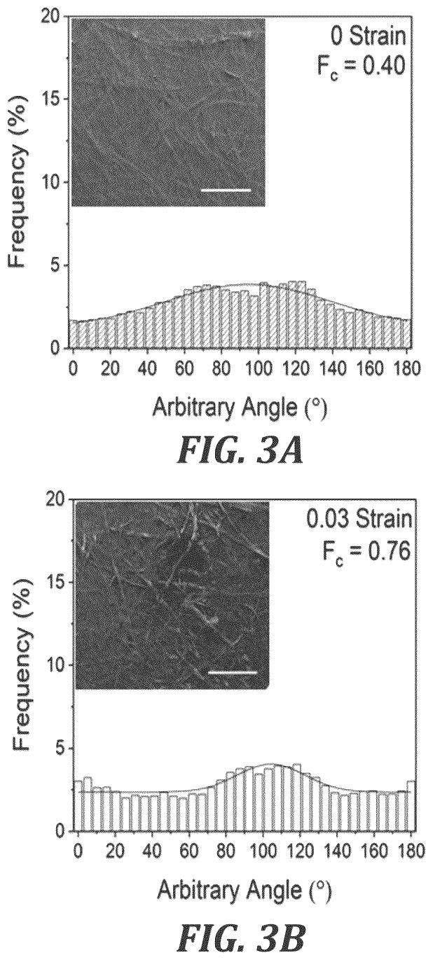

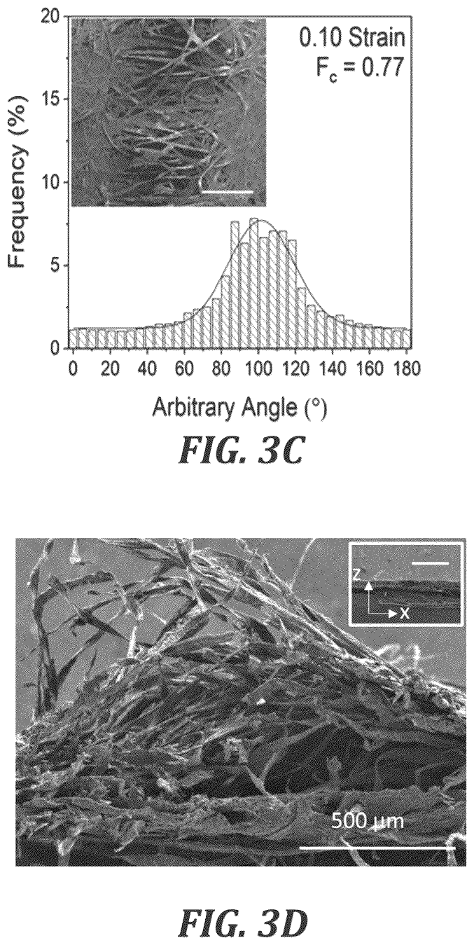

One of the auxetic mechanisms is the buckling of the out-of-plane fibers under a stretched random matrix. Due to the buckling, an extreme negative Poisson's ratio of −400 has been observed for individual fibers, as shown in FIG. 2B. This extreme auxeticity offers the capability of manipulating out-of-plane electrical junctions for resistance change. While conventional sensors made of positive Poisson's ratio show resistance increase upon pressure, the resistance of an auxetic material decreases due to the recovery of electrical connections. Such a piezo-resistive sensitivity is dramatically boosted by forming molecular junctions.

According to the percolation theory, a rapid increase of resistance occurs when strain becomes greater than a critical value. Beyond this threshold value, the percolated conductive network is drastically terminated to reduce the number of electrical paths in the material. In conventional materials, the disruption of the percolated conductive network is compensated by the reorganization of electrical paths in the out-of-plane direction due to Poisson's contractions. The piezoresistive sensitivity of auxetic materials can be amplified by the out-of-plane expansions in the auxetic structure. Furthermore, in response to a compressive load exerted on the surface, auxetic sensors exhibit a larger dynamic range in comparison to analogous conventional materials. Their superior sensitivity to strain makes the sensors particularly suited for delicate vibration monitoring, such as wrist pulse monitoring.

In some embodiments, the insulating fibers are compressed in the width direction and expanded out of plane with buckling to align fibers along the tensional direction as shown in FIG. 3D. As the cellulose fibers are realigned in the stretching direction, the fibers at the necking region may be compressed in the width direction, buckled, and forced against each other into the out of plane direction. As shown in FIGS. 3E-3F, the buckled cellulose fibers may exhibit ridges and valleys along the x-y place after fracture. As a result, thickness may be increased, resulting in a greater negative Poisson's ratio. In some embodiments, the thickness ranges from 80 to 120 micrometers.

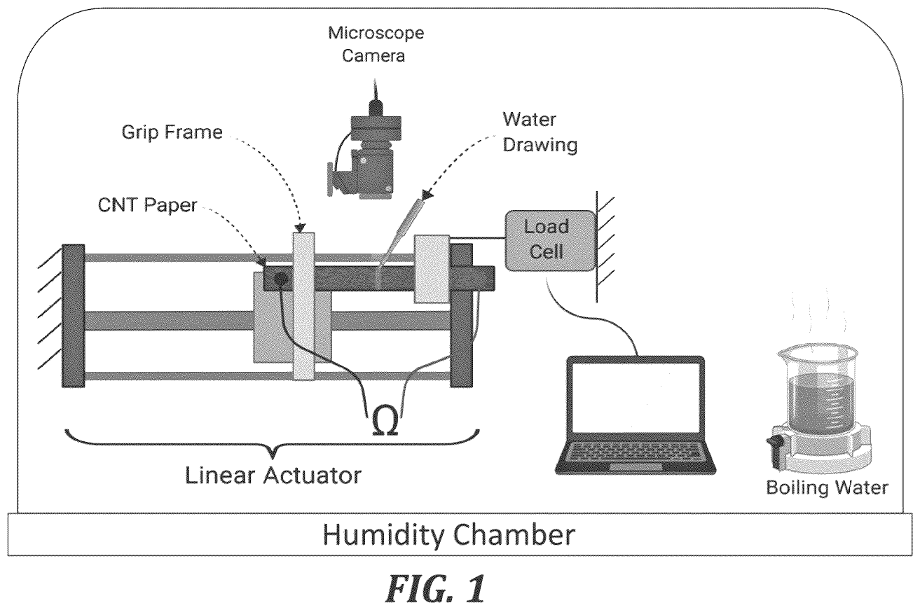

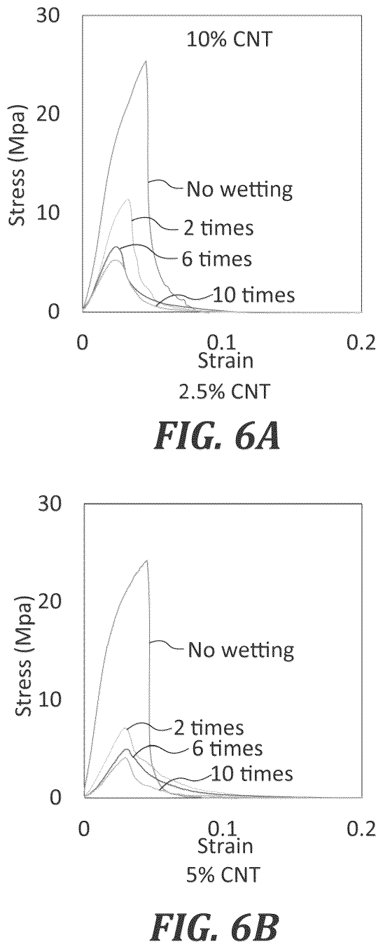

In some embodiments, a liquid is printed on the composite substrate before the sensor is stretched. In some embodiments, the liquid is printed onto the composite substrate to form a liquid printed region. FIG. 1 shows an example system for liquid printing under uniaxial tension to produce an example sensor. The liquid-printing method provides scalable fracture-induced fabrication of piezoresistive sensors based on a random network of cellulose fibers pre-adsorbed with CNTs, such as the CPC hand-sheets described above. Liquid printing can also further increase the negative Poisson's ratio, as shown in FIG. 2D. In some embodiments, the liquid printed region is a straight line. In some embodiments, the liquid printed region is a V, a W, a circular shape, or a random shape. An example of the V shape is shown in FIG. 4F. In some embodiments, the V shaped liquid printed region has a greater fracture area than the straight line liquid printed region. In some embodiments, the W shaped liquid printed region has an even greater fracture area than the V shaped liquid printed region. In some embodiments, the greater the fracture area, the greater the increase in sensitivity.

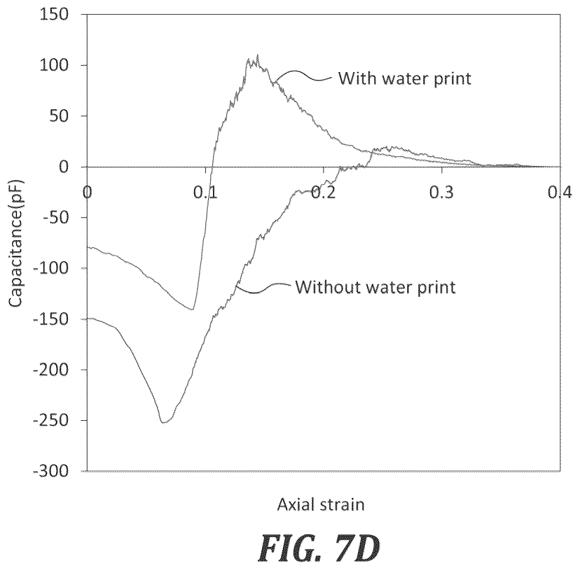

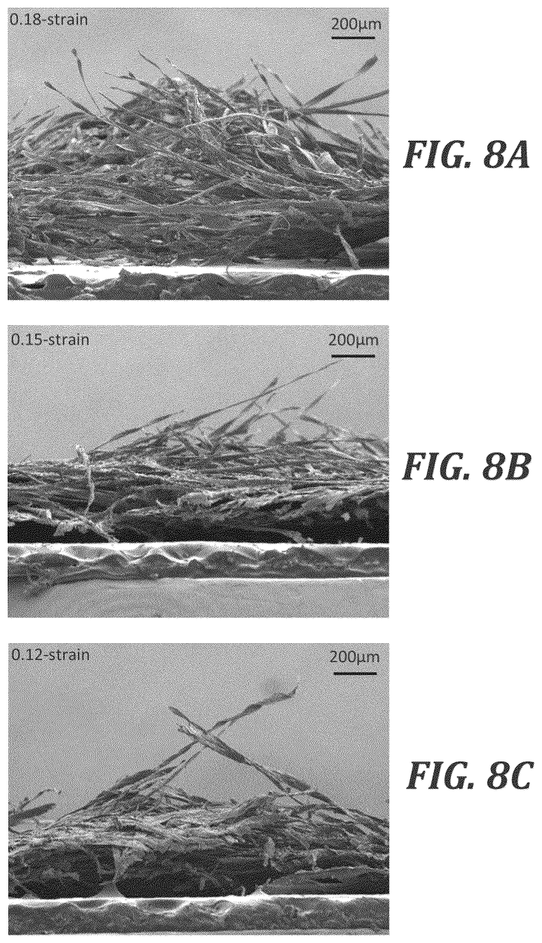

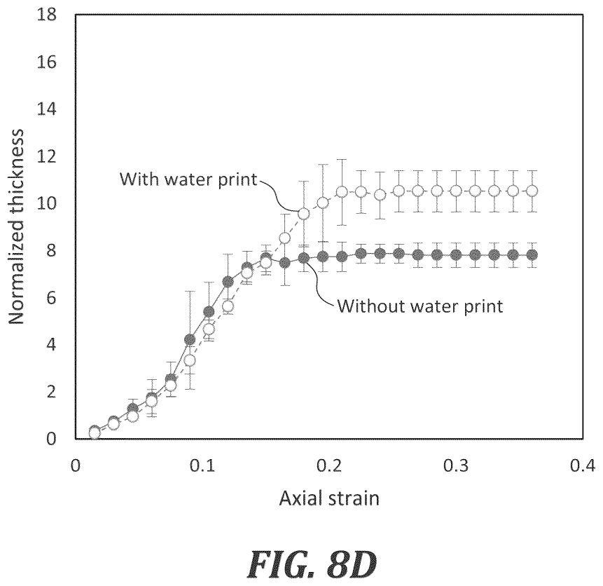

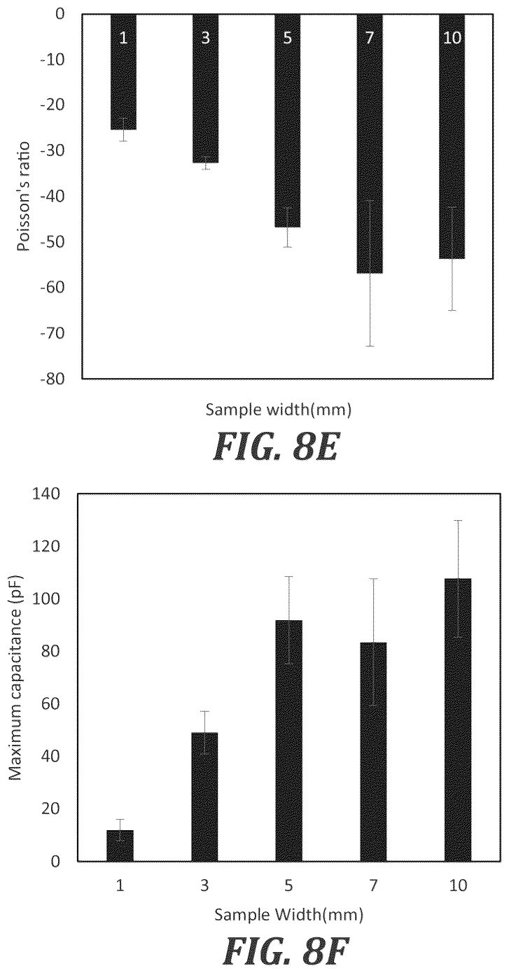

In some embodiments, the water printing is repeated, for a total of 2, 6, or 10 times. The repeated printing may lead to a reduction of the wet strength retention. In some embodiments, the wet strength retention is reduced by 35-45%. In some embodiments, the wet strength retention is reduced to 19-26%. In some embodiments, the insulating fibers are fractured along the liquid printed region to initiate and design a cracking pattern in the composite substrate. Examples of such designs are shown in FIG. 7B. To better control the fracture process, a noncontact liquid printing method may be applied to initiate the dissociation of cellulose fibers and the controlled cracking of CPC. In some embodiments, the liquid used in the liquid printing is water, but in other embodiments, the liquid may be any protic polar solvent, such as ethanol, acetic acid, or ammonia.

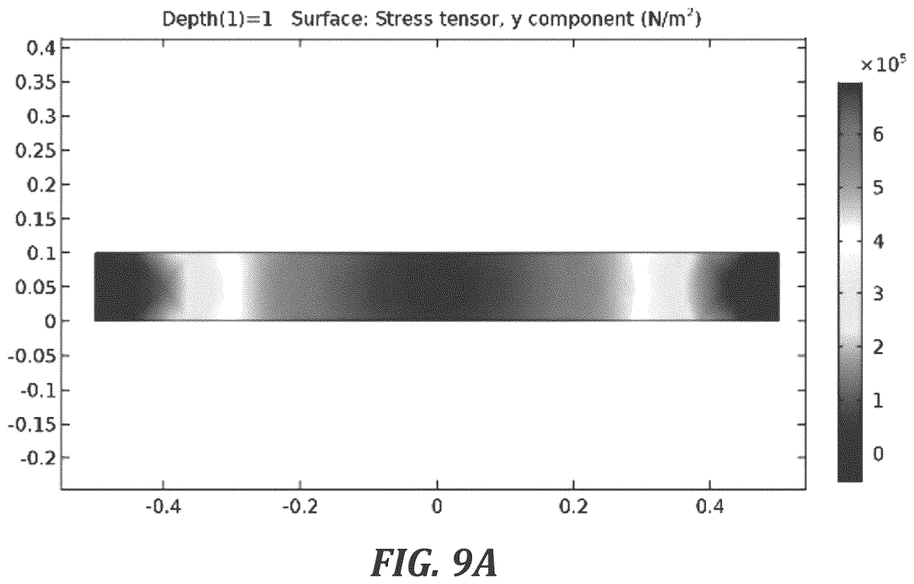

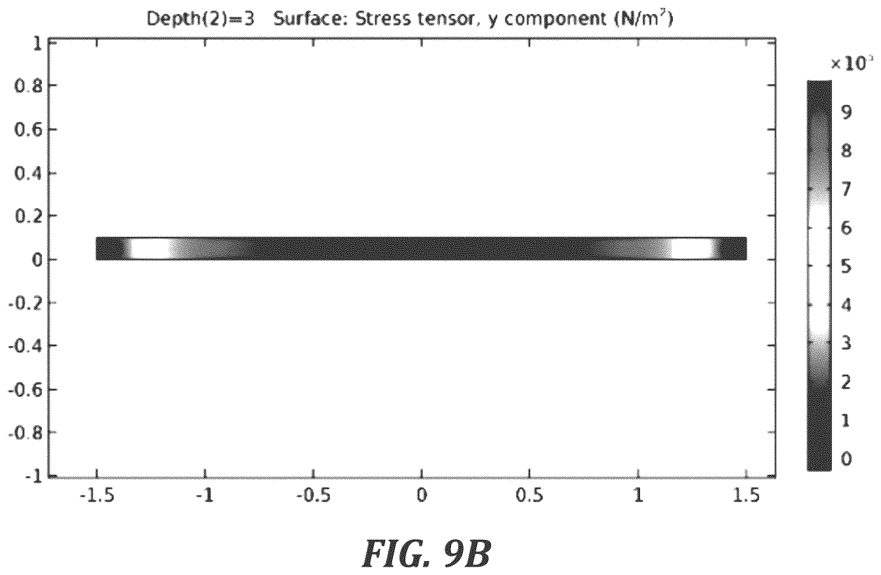

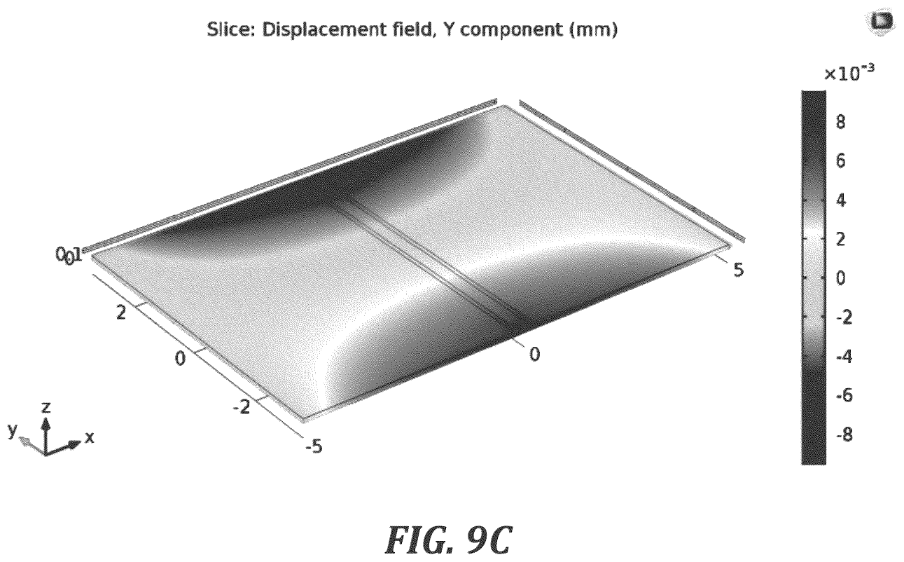

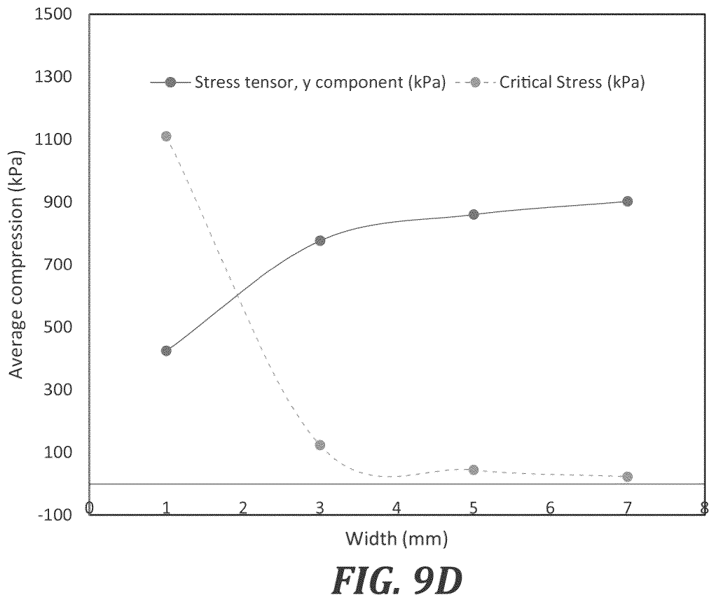

The auxeticity of the CPC is pronounced due to the stress concentration of varying elasticity and different Poisson's ratio in the dry-wet-dry CPC regions, as shown in FIG. 2C. The uniformly cracked and fractured CPC shows a remarkable resistive sensitivity. The resistive sensitivity may be produced through the percolation change under pressure. In some embodiments, the CPC is stretched at a strain between 0.1-0.24. In some embodiments, the strain is 0.18, 0.15, or 0.12, as shown in FIGS. 8A-8C. In some embodiments, the width of the fracture region ranges is up to 10 mm. In some embodiments, the larger stress in the x-direction was applied to the wet region, which results in the compression to the width direction (y-direction) with stretching. In some embodiments, the compression induces buckling, expanding the CPC in the z-direction.

In another aspect, a method of making a sensor comprising applying a unidirectional tensile force to the composite substrate, wherein the plurality of insulating fibers aligns along the tensile force and bulging with out-of-plane direction at the site of a tensional fracture, wherein the precursor composite substrate comprises a composite substrate comprising a template material, wherein the template material comprises a plurality of insulating fibers, and a plurality of carbon nanotubes bonded to the insulating fibers forming a nanotube coating on the insulating fibers, and a first electrode coupled to the nanotube coating on one side of the fracture and a second electrode coupled to the nanotube coating on the opposite side of the fracture, such that an electrical signal applied between the first electrode and the second electrode passes through the plurality of junctions at the site of the fracture is disclosed, as shown in FIG. 7A. CPC piezoelectric sensors may be fabricated with the method of controlled liquid printing and stretching of the CNT composite papers. In some embodiments, the liquid printing is non-contact liquid printing. In some embodiments, the liquid is water. Water may be printed onto the CNT composite papers with a liquid bridge printing method, where constant water volume is supplied by maintaining a consistent contact angle and printing speed. After the liquid printing, the CNT composite may be stretched so as to create a fracture. In some embodiments, the fracture is a fractured region, where fibers buckle out of plane in response to the stretching. The CNT composite papers are stretched until the fibers buckle and create a fracture, but not so far as to sever the CNT composite paper, i.e., the CNT composite paper remains connected, as shown in FIG. 3F. FIG. 7B further illustrates the fracture. In some embodiments, the fracture-induced buckling of cellulose fibers by water-printing exhibits localized and predictable behaviors of the fibers due to the selectively reduced strength of inter-fiber junctions and the stress concentrations. In some embodiments, the fibers are fractured along the liquid printed region under a high relative humidity environment having a humidity between about 80% to 100% humidity. In some embodiments, the humidity is 95% for extended stretching. In some embodiments, the liquid printing is repeated under low humidity environment having a humidity between 0 to about 80% humidity in order to make the composite fully wet, as shown in FIG. 2C.

The CPC may be locally fractured with necking along a region due to the reduced CPC strength and stress concentrations. Due to the wetting—stretching method, the fracture process of CPC may be reproducibly manipulated with six-time water-printing. The amplified auxetic behavior is a result of the buckling of wet CPC matrix during fracture. The auxetic behavior of CPC improved the piezoresistive sensitivity through the recovery of terminated electrical pathways upon applied pressure.

In some embodiments, the liquid printing produces a plurality of high aspect ratio cantilevered structures along the printed region. In some embodiments, the plurality of cantilevered structures are aligned along the tensional direction. The auxetically modified CPC can change capacitive junctions. The molecular junctions of cellulose fibers embedded with CNTs create capacitance. The buckled structure produces cantilever-shaped electrodes to form a capacitive sensor. Compared to traditional strain and pressure gauges, novel electromechanical coupling mechanisms, such as disconnection of sensing elements, tunneling effect, and fracture-induced sensitivity optimize the sensitivity of piezoresistive materials.

The capacitive response of wet-fractured carbon nanotube composites may further be applied for use in humidity. The stretched composite strip may be fractured and buckled in the width to show numerous radial cantilevers consisting of cellulose fibers coated with carbon nanotubes. The composite fibers form molecular junctions to significantly increase capacitance under high humidity. The molecular junctions switch electric current flow between resistance and capacitance. The resulting capacitive sensor works as a humidity sensor detecting humidity without any absorption medium. The novel auxetic behavior of a composite paves the way for inexpensive humidity and sweat sensors.

In some embodiments, the liquid printing increases the surface area of the composite substrate. Due to the large surface area and a high electric field of auxetically created structures, the capacitive junctions can be sensitive to humidity change. The water molecules introduced to the fibrous junctions can increase a sensitivity to humidity. The sensing response to humidity may be compared to a commercial humidity sensor for sweat detection.

In some embodiments, the sensor is prepared by fracturing the CPC sensor as described above, and further laminating the sensor. In some embodiments, the sensor is laminated with 20 μm-thick polyester film.

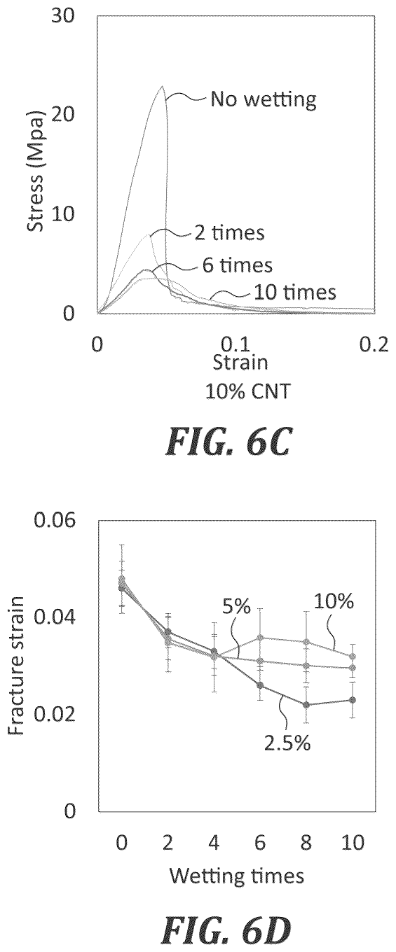

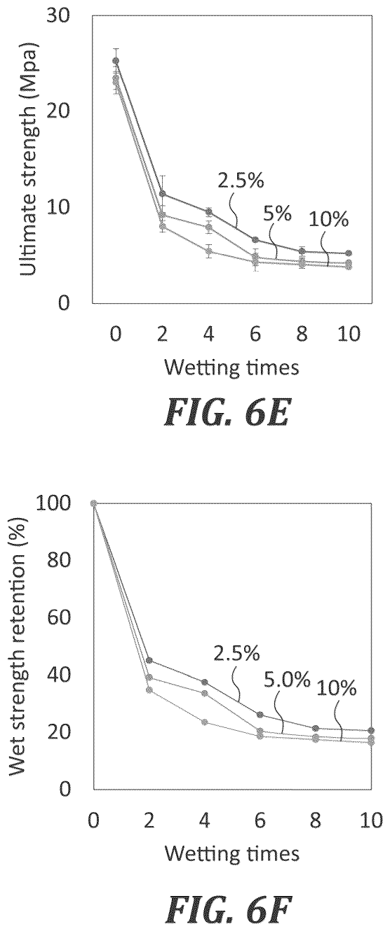

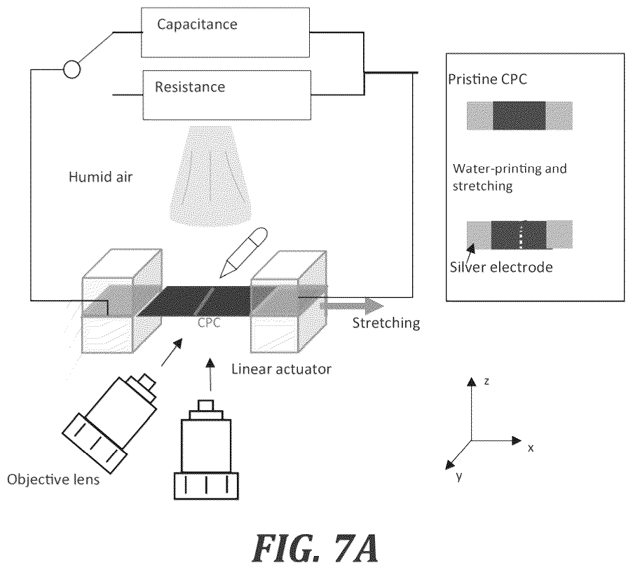

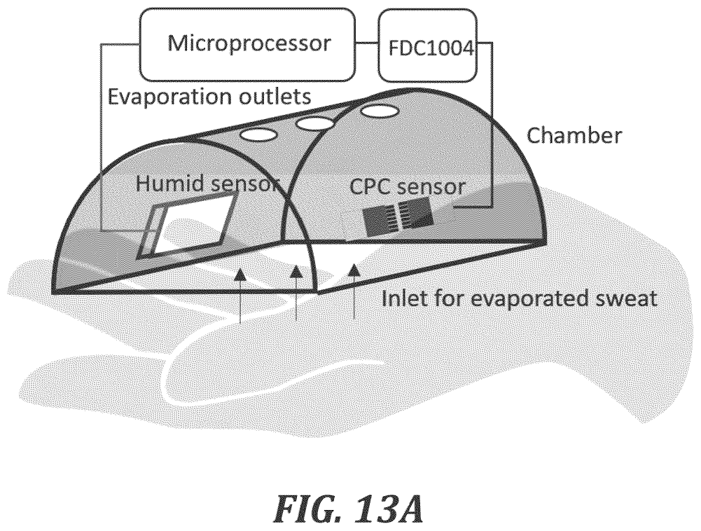

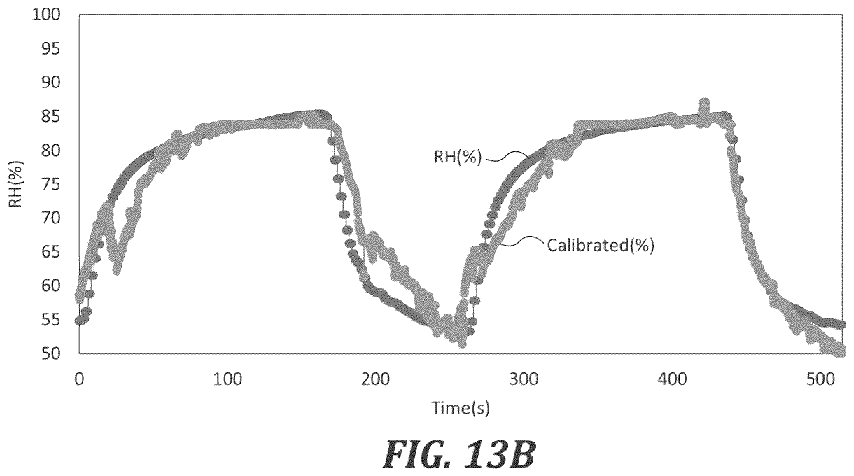

In another aspect, a sensor manufactured by any of the methods described herein is disclosed. The sensor may be used in a variety of applications, including humidity sensing, as illustrated in FIG. 13A, and step counting, as shown in FIG. 5A. As illustrated in FIG. 13A, the sensor may be comprised of CPC that is stretched so that the fibers within the sheet buckle and align within the tensional direction. In some embodiments, the sensor is an in-plane strain sensor, an out-of-plane piezo-resistive sensor, or a capacitive sensor. In some embodiments, the sensor is a heartbeat sensor, a gripping motion sensor, a breathing sensor, a nasal air flow sensor, a finger movement sensor, a proximity sensor, or a human-machine interface. In some embodiments, the sensor is a humidity sensor configured to measure humidity and environmental gas composition change. In some embodiments, the sensor is a bistable resistance-capacitance component that is controlled by humidity.

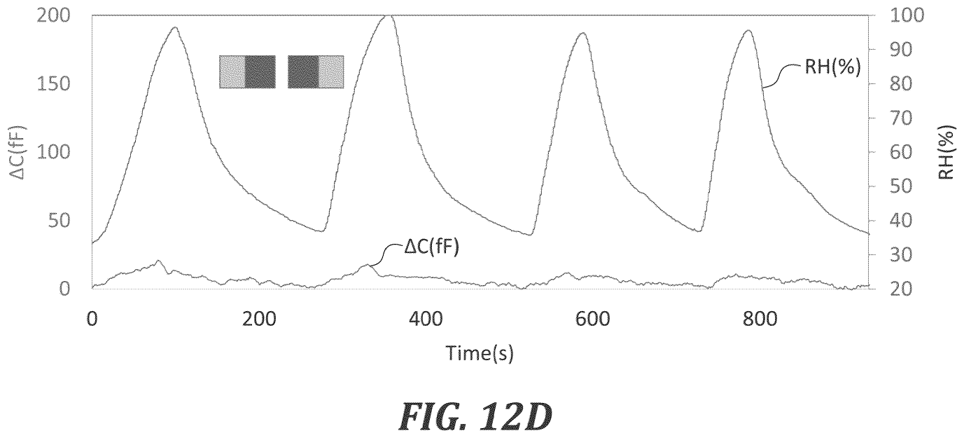

In some embodiments, the CPC sensor is sealed to avoid damaging the sensing element. In some embodiments, the CPC sensor is sealed with polyethylene terephthalate (PET) film, as shown in FIG. 12C. FIG. 5A is a CPC piezoelectric heartbeat sensor capable of measuring the rate of cardiovascular pulsations when wrapped around the wrist of an individual, and FIG. 5B depicts a CPC piezoelectric sensor on a belt. In some embodiments, the cyclic motion from thoracic or abdominal expansions and contractions during inhalation and exhalation may be detected by mounting a CPC piezoresistive sensor on a belt. In some embodiments, the belt tension is adjusted such that the respiration motion could generate adequate relative pressure. Tailoring auxeticity of a random matrix paper-based composite offers a new route to enhancing the piezoresistive sensitivity with the improved manufacturing reproducibility toward wearable applications, for instance, the gait and respiration detection.

As mentioned above, the capacitive sensing mechanism of the fractured CPC may be used for humidity testing. In some embodiments, the high aspect ratio of the cellulose fibers created by axial stretching enhance the electric field around the crack domain. In some embodiments, water molecules are introduced on the surface of the crossing radial structure to enlarge the capacitance change among the high aspect ratio electrodes, resulting in an extreme change of capacitance. When the fibers are exposed to water vapor, the water molecules may absorb on the surface area where a high electric field is produced to form capacitance.

In some embodiments, the sensor may be used to measure humidity on a hand, as shown in FIG. 13A. In some embodiments, this device may include an evaporation hole and a CPC sensor. In operation, when the CPC sensor is placed on the palm, sweat evaporation may be measured on the hand. By using the CPC sensor described above, the capacitive change to humidity is significant without an absorption medium, and the air permittivity change due to humidity is negligible. The major capacitive response is the result of the change of CNT surface on the cellulose fibers coupled with a high electric field.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

Example devices, methods, and systems are described herein. It should be understood the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood other embodiments may include more or less of each element shown in a given Figure. Further, some of the illustrated elements may be combined or omitted. Yet further, an example embodiment may include elements not illustrated in the Figures. As used herein, with respect to measurements, “about” means +/−5%.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The present disclosure may be better understood with reference to U.S. patent application Ser. No. 16/768373, “Fiber-Based Composite With Fracture-Induced Mechano-Electrical Sensitivity,” the disclosure of which is hereby incorporated by reference in its entirety.

The following examples are included for the purpose of illustrating, not limiting, the described embodiments.

### EXAMPLES

### Example #1

### Materials

Bleached Kraft softwood pulp (SW) was kindly provided in a dried mat form from Port Townsend paper mill. Alkali lignin (AL, 99%), sodium dodecyl sulfate (SDS, 99%), and cationic polyacrylamide (CPAM, Percol 3035) were obtained from Tokyo Chemical Industry Co., MP Biomedicals, and BASF, respectively. Hydroxyl-functionalized carbon nanotubes (CNT-OH), synthesized from catalytic chemical vapor deposition, were purchased from Cheap Tubes Inc. As per the manufacturer data, CNT-OH have lengths in the 10-20 mm range and mean diameter of 50 nm, with an average of 5.5% of OH groups. All chemicals were used as received without any additional treatment.

### CPC Preparation

CNT-cellulose composite papers were prepared following a modified TAPPI T-205 standard method, as previously reported elsewhere. Briefly, hand-sheets were formed by a filtration method using a hand-sheet molder (Essex International Inc. Custom Machinery), and pressed and dried according to TAPPI T-205 standards. Prior to sheet formation, CNT-OH were dispersed in a binary mixture of AL and SDS (90 : 10 wt) using a double acoustic irradiation system, to promote individualization in solution and achieve a uniform distribution of charge transport routes throughout the final composite.38 Aqueous dispersion of CPAM were first added to pulp fiber solutions (0.3% consistency) and combined on a hot plate at 50° C. for 30 minutes. The as-dispersed CNT-OH solutions were then added to the pulp mixture and kept under constant agitation for 30 minutes. The combined CNT-OH and pulp suspensions were then filtered, pressed, and dried to form hand-sheets. The proportion of cellulose fibers, CNTs, CPAM, AL, and SDS were adjusted to achieve a total mass of 1.2 g OD (60 g m⁻²).

For comparison purposes, hand-sheets were also prepared without any CNT-OH, just using a pulp/CPAM/AL/SDS blend and denoted as “control” samples. All hand-sheets were kept for 48 hours under room temperature conditions (23° C.) and 50% relative humidity prior to testing. All hand-sheets had a mean thickness of 88.4-3.1 mm. Fabrication of the CPC piezoresistive sensors by water-printing CPC piezoresistive sensors were fabricated by controlled water-printing and stretching. Silver paste (MG Chemicals, USA) was applied to both ends of the CPC strip and cured at 70° C. on a hot plate to make the electrodes. The water was printed on CPC specimens by using a noncontact printing method. Using a liquid bridge printing method, constant water volume was supplied each run by maintaining a consistent contact angle and printing speed. Using a 0.8 mm-diameter capillary pen, water was printed repeatedly with a 3-dimensional controller.

FIG. 1 s a system for water printing under uniaxial tension to produce auxetic CPCs, in accordance with the present technology. The stretching was applied by a tensile test stage in a humidity chamber. A boiled water source was supplied in the chamber to maintain a humidity of 80% at 28° C. FIG. 1 shows the CPC before and after the water-printing and stretching process. controlled environment, the printed water volume on the CPC was kept constant during the test by preventing evaporation. In operation, the CPC is placed on a linear actuator, between the grip frames. Water is drawn onto the CPC, and the CPC is stretched.

Regarding the stretching test, the strain was defined by:

$$ε = \frac{L - {L}_{0}}{{L}_{0}}$$

where L is the length of the specimen under stretching, and L₀ is the original length (10 mm) of the specimen.

The fracture strain was defined to be the strain at the fracture under stretching. For reproducible fabrication procedures, force and resistance were recorded by a load cell (DYMH-103, CALT, China) and a multimeter (Fluke Corp., USA), respectively. The stress was calculated by:

$$σ = \frac{F}{D \times T}$$

where F was the force measured by the load cell, D was the pristine width of the specimen, and T was the thickness of the specimen measured by a digital gage (PK-0505, Mitutoyo, Japan).

### Auxetic Behavior Characterization

The auxetic behavior of the CPC was studied by measuring the thickness changes. In the setup of the CPC sensor fabrication stage, as shown in FIG. 1, a stereo zoom microscope was focused on the region of the specimen from the transverse direction of the stretching. The thickness change of the specimen during the water-printing and stretching was measured using optical microscope images and ImageJ software. The measured thickness was also validated by a scanning electron microscopy (SEM, XL830, FEI Company, Hillsboro, OR, USA) study. The thickness was compared for pure paper, and CPC with the CNT concentrations of 2.5, 5, and 10 wt %. The instantaneous Poisson's ratio (Vᵢₙₛₜ) and effective Poisson's ratio (V_{eff}) were computed based on the following equations:

$${V}_{inst} = \frac{( {z}_{i} - {z}_{i - 1} ) / {z}_{i - 1}}{( {l}_{i} - {l}_{i - 1} ) / {l}_{i - 1}} \qquad ( 1 )$$

$${V}_{eff} = \frac{( {z}_{i} - {z}_{0} ) / {z}_{0}}{( {l}_{i} - {l}_{0} ) / {l}_{0}} \qquad ( 2 )$$

where lᵢ and zᵢ denoted the specimen length and thickness values at the given strain level, and lᵢ−1 and zᵢ−1 denoted their values at the previous level. l0 and z0 denoted the original specimen length and thickness. 10 was 10 mm, containing the wet, semi-wet and dry regions after water-printing. For the specimens with the CPC six-times, CNT contents were 0, 2.5, 5, and 10 wt %. Vᵢₙₛₜ was computed at the strains of 0.02, 0.03, 0.04, 0.05, 0.08 and 0.10. V_{eff} of six-time water-printed paper and CPCs were computed at strain of 0.01, 0.02, 0.03, 0.04, 0.05, 0.08 and 0.10. The V_{eff} of non-water-printed paper and CPCs were computed at their fracture strain.

The V_{eff} of non-specimens at fracture strain was compared with that of six-time specimens that had maximum magnitudes.

While the elastic theory constrains the Poisson's ratio to a range between −1 and 0.5, 5 a computational study reported an in-plane Poisson's ratio of −17 for an auxetic structure comprising rotachiral lattices. Additional research provided further guidelines to design auxetic structures with large Poisson's ratio based on a programmed geometric layout for highly deterministic and periodic structures.

### Characterization of the Anisotropy of Fractured CPC

SEM (XL830, FEI Company, Hillsboro, OR, USA) was used to study the CPC surface morphology in-plane and fracture length. The CPC 2.5 wt % was sputter-coated with gold/palladium with the thickness of 6-7 nm. To ascertain the fracture length and morphology, the CPC was mounted to a flat aluminum stage using carbon tape and imaged using a 5 kV accelerating voltage with a 5 mm working distance. Fracture length and pulp fiber orientation were determined using Image J software and the following equation:

$${f}_{c} = \frac{F W H M - 180}{180} \qquad ( 3 )$$

FWHM represented the full width half maximum of the peak created from the Gaussian fit conducted on the alignment histogram. UV-vis measurements were performed on a PerkinElmer Lambda 750 spectrophotometer equipped with a 100 mm-integrating sphere operating in the 450-850 nm range. CPC samples were mounted on top of a 3 mm-diaphragm, and a polarizer was used to capture anisotropy. For the convenience of the discussions of orientations, the stretching direction was defined to be x-direction, the in-plane direction perpendicular to x-direction was y-direction, and the out-of-plane direction was z-direction. Determined by their angles to x-direction, cellulose fibers in x-z plane were categorized into tilted and inclined fibers.

### Characterization of Piezoresistive Sensitivity

Piezoresistive force sensors were fabricated by the water-printing and stretching method. CPC with 0, 2.5, 5, and 10 wt % CNT were used with the six times of water-printing and 0.1-strain. A straight water line was printed on the CPC samples. The piezoresistive sensitivity was characterized by a PDMS block integrated with a load cell as shown in FIG. 1. The dimension of the PDMS block was 7×15×2 mm3 to completely cover the fracture area (approximately 1.5×5 mm2). The linear actuator was controlled to apply the repeated force between 0 and 500 kPa at a speed of 55 mm s⁻¹. A multimeter was connected to the sensor to measure the resistance change when the sensor was pressed. The sensitivity of the sensor was S=(ΔR/R₀)/Δp, where ΔR was the resistance change of the sensor, R₀ was the initial resistance of the sensor, and Δp was the change of the applied pressure. The error bars were calculated to study the reproducibility of the sensor's sensitivity. The dynamic range (DR) was defined as

$$D R = \frac{{P}_{high}}{{P}_{low}} ,$$

where P_{high} and P_{low} were the highest and lowest pressure that could be measured by a sensor.

To demonstrate sensor applications, heartbeat, respiration, and gait movement were measured. The sensors were tested by de-identified volunteers. Among the testing results from multiple volunteers, a randomly chosen secondary data set was demonstrated for sensing performance evaluation.

### Results and Discussion

Tailored auxetic behavior of CPC for sensor fabrication. The water-printing method provided scalable fracture-induced fabrication of piezoresistive sensors based on a random network of cellulose fibers pre-adsorbed with CNTs, as shown in FIG. 1. Pristine CPCs consist of randomly oriented pulp fibers embedded with well-dispersed CNTs with no obvious aggregations. The water-printing applied by a capillary pen enabled the non-contact wetting with a desired pattern. Water ink was supplied through an ink bridge formed between the pen nib and the substrate. The water-printing flow rate was kept constant among all samples through control of the pen tip height from the substrate, contact angles, and printing speed. Using the liquid-bridge printing method, accurate water lines could be printed repeatedly without damaging the substrates. After the water-printing was repeated six times, the sample was stretched until fracture and its electrical resistance was recorded. The strain was applied until 0.3 where the stress magnitude for all stretched specimens became 0. The electromechanical coupling of CPC prepared at various CNT loadings (i.e. 2.5, 5, and 10 wt %) was studied during uniaxial stretching. It can be observed that the onset of resistance changes due to in-plane stretching corresponds to the largest variations in the instantaneous Poisson's ratios.

FIG. 2A is a graph showing the stress-strain relationship coupled with normalized resistance change, in accordance with the present technology. On the left-side vertical axis is stress in MPa. On the horizontal axis is strain. On the right-side vertical axis is the normalized resistance. The stress and normalized resistance is labeled.

FIG. 2B is a graph showing Instantaneous Poisson's ratios of pure paper and CPC with CNT wt % of 2.5, 5 and 10 during stretching, in accordance with the present technology. Also shown are optical images of CPC profiles with 2.5 CNT wt % before and after fracture (e=0.02 and 0.10). The original CPC thickness is 100 mm. On the vertical axis is the Instantaneous Poisson ratio, and on the horizontal axis is the strain.

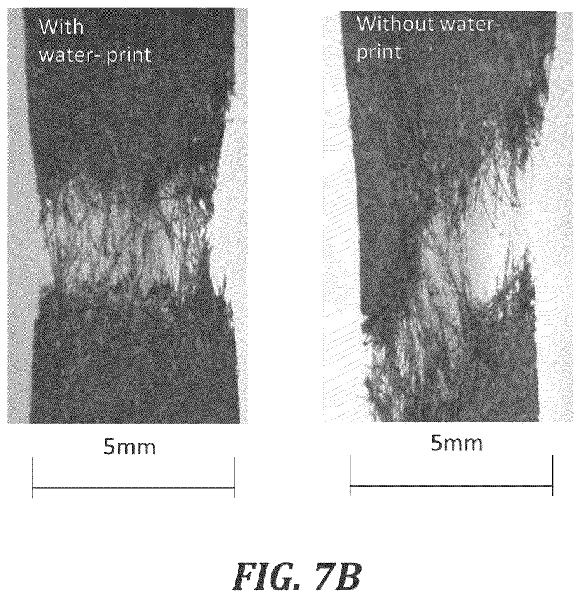

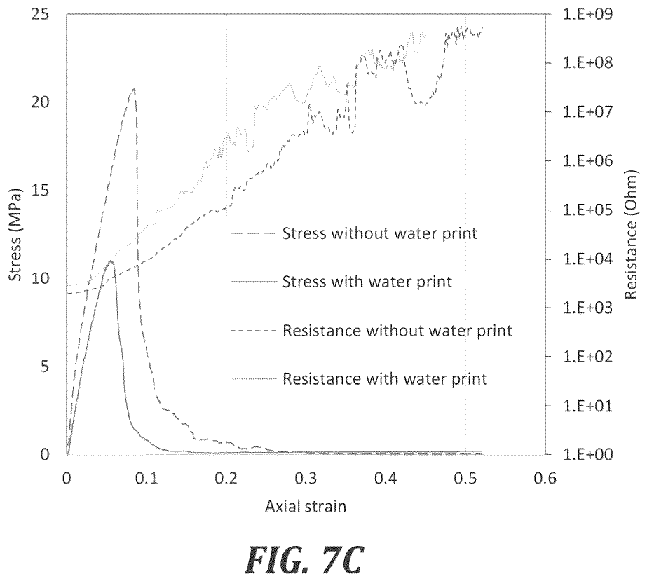

This electromechanical coupling offers a simple method to streamline the manufacturing of auxetic CPC by measuring the electrical resistance. The mechanical properties of the composites that are for 0, 2, 6, and 10 times are presented for the CPC with all three CNT contents in FIGS. 6A-6F. As the indications of reproducibility of mechanical properties of water-printed CPC, the fracture strain, ultimate strength, and wet strength retention of CPC of 2.5, 5, and 10 wt % were demonstrated with the water-printing times of 0, 2, 4, 6, 8 and 10. The fracture strain and ultimate strength were reduced by the increased number of water-printing, and meanwhile the reproducibility increased. For the CPC of 2.5 wt %, the fracture strain and ultimate strength were 0.026±0.0031 and 6.6±0.11 MPa with six times of water-printing, and 0.04±0.0037 and 25±1.3 MPa without water-printing. The reduced deviations of the ultimate strength were obtained by the localized, predictable wet fracture process of CPC.

To eliminate the effect of strength difference among non-CPC with different CNT contents, the strength reduction by water-printing on CPC was reflected by their wet strength retention. The wet strength retention was defined as the ratio of the average ultimate strength of CPCs to that without water-printing. A two-time water-printing significantly reduced the wet strength retention to 35-45%. The strength reduction began to saturate at six-time water-printing, when the wet strength retention reached 19-26%. Therefore, the six-time water-printing was selected for fracture manipulation. The CPC with higher CNT wt % showed the lower wet strength retention, indicating that the water-printing method had the greater reduction of CPC strength with the lower CNT content. This was attributed to the greater hydrophilicity with more hydroxyl functionalized CNTs embedded on cellulose fibers, which was supported by the contact angles. The different wetting characteristics were demonstrated by contact angle measurements averaged over six replicates. The 2.5 wt %-CPC and 10 wt %-CPC yielded the contact angles of 91.5±0.71 and 88.5±0.51, respectively, resulting in a greater diffused wet area at higher CNT contents. This observation was consistent with the fractured length determined at various CNT content and under the same applied strain.

The electromechanical properties of CPC were depicted as a two-stage resistance response, including the slow increase of resistance before an inflection point followed by the rapid increase. The inflection point was declared when the stress—strain curve deviated from the linear slope by 5%. The two stage increase of the resistance was dominated by the breakage of CNTs spanning the cellulose fibers, and the fracture-induced rapid reduction of tunneling effects, respectively. The slow and rapid resistance increases at low and high strains were qualitatively consistent with the piezoresistive properties of other CNT composites. The normalized resistances at 0.3-strain were 27.3, 18.7, and 10.1 for the CPC with 2.5, 5, and 10 CNT wt %, respectively. The higher normalized resistance of CPC with lower CNT content after fracture indicated less retained electrical paths. It was discovered that the water-printing amplified and localized the out-of-plane directional auxeticity of CPC by predictable fractures. The thickness view of the optical microscope images showed that the auxetic behavior of random CPC networks was locally induced by the controlled fracture developed via the water-printing method.

The instantaneous Poisson's ratio (Vᵢₙₛₜ) was measured to assess the auxetic behavior of CPC at representative strain values, as shown in FIG. 2B, which indicated the instantaneous increase of the specimens' thickness at certain strains. The Poisson's ratio ranged from −0.26 to −0.19 in the elastic range (strain<0.02), where the fibers were forced to expand the thickness in the transverse direction due to the stretching. The drastic augmentation in thickness occurred at the plastic deformation range of 0.03-0.04 strain, indicated by the highest magnitude of Vᵢₙₛₜ, which was synchronized with CPC's stress increase in FIG. 2A. The Vᵢₙₛₜ remained negative until strain of 0.10, indicating the continuous increase of the thickness. When the applied strain was greater than 0.10, Vᵢₙₛₜ became 0. Due to the instant, unpredictable fracture process, Vᵢₙₛₜ for non-specimens was not measured. The localized and predictable fractures by water-printing opened a way to study the mechanical properties and plastic deformation of random network structures.

FIG. 2C shows simulation results of stress distribution for CPC under tension, in accordance with the present technology. The inset shows the entire CPC in context, while the larger image is a closeup of the CPC. On the right side is a scale denoting pressure in 10 MPa. The localized fracture of CPC was attributed to the reduced strength of cellulose fibers and the stress concentration induced by water-printing, as shown in FIG. 2C. Finite element analysis (FEA) demonstrated that the stress was concentrated at the semi-wet region between the fully wet and dry regions of CPC due to the localized auxetic behavior and the different stiffness of wet and dry CPC. To assess the contribution of auxetic behavior and stiffness difference to the stress concentration, the stress concentration factor (Kₜ) was defined as the ratio of the maximum stress (σₘₐₓ) to the stress without auxetic behavior and stiffness difference (σ₀). When only the auxetic behavior was considered in the numerical analysis, Kₜ was 1.3. Considering only the stiffness difference, Kₜ was 1.4. Due to the numerical errors resulting from the large magnitude of negative Poisson's ratio, the simulation was conducted in the small strain range below 0.02. The applied strain and the magnitude of Poisson's ratios in wet and semi-wet regions were much smaller than those under fracture. Apparently, Kₜ will increase as the difference of Poisson's ratios enlarge. In combination with the stress concentration at the semi-wet region, the fracture was initiated at the center of the wetted region due to the significant strength reduction of the wet CPC. The necking showing the reduced width occurred at both the semi-wet and fully wet regions.

The remarkable auxeticity was induced by fracture and enhanced by the water-printing. The auxeticity of a specimen was indicated by V_{eff}, which showed the averaged Poisson's ratio from 0 to a certain strain level. The maximum V_{eff} magnitudes (V_{effmax}) of specimens were obtained slightly after fracture (strain=0.04-0.05). Since the V_{effmax} indicated the greatest auxeticity of a CPC specimen under stretching, the magnitude of V_{effmax} was chosen for the quantitative comparison of auxeticity of paper and CPC specimens. The maximum thickness and V_{effmax} of non-water-printed specimens were obtained at the fracture (strain=0.02-0.03).

FIG. 2D shows the maximum effective Poisson's ratios for water-printed and non-pure paper and CPC with CNT wt % of 2.5, 5, and 10, in accordance with the present technology. Also shown are optical microscope images of fractured profiles of CPC with 10 CNT wt % with and without water-printing. On the vertical axis is the Effective Poisson ratio at maximum magnitude, and on the horizontal axis is the CNT weight in percent. The effect of the water-printing process on the auxetic behavior of the fibrous composites was assessed by comparing the V_{effmax} of and non-specimens. The V_{effmax} values of paper and CPC with 2.5, 5, and 10 CNT wt % were significantly greater than their fully dry counterparts by 2.6, 2.5, 2.5 and 2.3 times. It was also found that the V_{effmax} values of the fully wet CPC were 1.9, 1.9, 1.8 and 1.7 times those of the non-counterparts. Regardless of water-printing, the lower CNT contents consistently yielded the more pronounced auxetic behavior. For instance, the V_{effmax} of 2.5%-CPC was −49.5, which was 1.09 times that of 10%-CPC. Remarkably, the V_{effmax} of paper raised to −56.7 in the absence of CNTs.

### Mechanisms of Fracture-Induced Auxetic Behavior of CPC

To understand the underlying mechanism of fracture-induced auxetic behavior tailored by water-printing, SEM study was conducted at various representative stages of stretching to investigate in-plane and out-of-plane orientations of fibers as shown in FIGS. 3A-3D.

FIGS. 3A-3C are SEM images and fiber orientation of 2.5 CNT wt %-CPC at strain of 0, 0.03, and 0.10. On the vertical axes is the frequency in percent, and on the horizontal axes is an arbitrary angle in degrees.

FIG. 3A is a SEM image and fiber orientation of 2.5 CNT wt %-CPC at strain of 0, in accordance with the present technology. FIG. 3B is a SEM image and fiber orientation of 2.5 CNT wt %-CPC at strain of 0.03, in accordance with the present technology. FIG. 3C is a SEM images and fiber orientation of 2.5 CNT wt %-CPC at strain of and 0.10, in accordance with the present technology.

FIG. 3D is SEM image of fractured CPC with 10 CNT wt % at strain of 0.10 in accordance with the present technology. The scale bar indicates 500 μm.

The fiber orientation within stretched CPC of 2.5 wt % was plotted at 0, 0.03, and 0.10 strain. The orientation factor, fc, ranging from 0 (fully isotropic) to 1 (perfect alignment) was determined. Localized at the fractured region of the specimens, the fiber alignment to the stretching direction increased under applied strain, regardless of CNT contents.

The observation was consistent with polarized absorption spectroscopy data confirming the optical anisotropy was observed only at the fractured region of the strained samples. The CPC with the lowest amount of CNT (i.e. 2.5 wt %) exhibited the highest degree of fiber orientation, with a fc of 0.77 at a strain of 0.10, and the largest auxeticity with a Poisson's ratio of −31.0. The significant fiber reorientations in z direction were verified by SEM images. Different from the compact layers within a pristine cellulose fiber matrix, the fractured CPC showed larger inter-fiber distances. After the fracture, broken cellulose fibers at fractured region lifted towards z direction and formed larger angles to the x-y plane, exhibiting much larger thickness at fractured region.

FIG. 3E is a SEM image of a pristine CPC, in accordance with the present technology. The of fracture-induced x-z planar structure reorganization under stretching is shown. The scale bar shows 1 mm. The optical images showing the in-plane and out-of-plane geometries at the fractured regions on the CPC were shown in FIG. 3E.

From the in-plane view, necking was observed at the water-printed region on CPC, where the thickness was also the largest, as observed in the out-of-plane view. The smallest width of the fracture region was 3.8 mm, which was reduced by 23% compared to the original width.

The remarkable auxetic behavior of CPC resulted from the buckled fibers in the cellulose network under localized fracture. Upon fracture initiation, some inter-fiber junctions were weakened by water-printing and thus more readily disrupted. The breakage of the hydrogen bonds between cellulose fibers allowed for higher mobility of randomly distributed fibers, as demonstrated by the increasing fc with applied strain as shown in FIGS. 3A-3C. As these random cellulose fibers were realigned in the stretching direction (x axis) by fracture, cellulose fibers at the necking region were compressed in the width direction, as shown in FIG. 3E, buckled, and forced each other into out of plane direction which is illustrated in FIG. 3F.

FIG. 3F shows cellulose fibers buckled, and forcing each other into the out of plane direction, in accordance with the present technology. Numerous buckled cellulose fibers exhibited ridges and valleys along the x-y plane after fracture.

As a result, the thickness was increased, resulting in a greater negative Poisson's ratio. The numerical and experimental results supported the auxetic mechanism. According to the stress concentration, the larger stress in x-direction was applied to the wet region, which resulted in the compression to the width direction (y-direction) with stretching. The compression-induced buckling expanded the CPC in z-direction. According to numerical simulation, in-plane necking and out-of-plane bulging were observed. Experimentally, the bright and dark contrast of the cellulose fibers in the SEM images clearly shows the ridges and valleys in the inset. The spike of Vᵢₙₛₜ at CPC's fracture strain also indicated that the auxeticity was induced by the buckling of cellulose fibers at fracture as shown in FIG. 2A. Unlike the buckling of individual fibers in a previous report, the fracture-induced buckling of cellulose fibers by water-printing exhibited the localized and predictable behaviors of fibers' due to the selectively reduced strength of inter-fiber junctions and the stress concentrations.

According to the orientation factors and Poisson's ratios of CPC with different CNT contents, the higher CNT contents led to reduce in-plane realignment of cellulose fibers, as well as decrease auxeticity. As a decisive factor of the auxetic behavior, the buckling of the cellulose fibers required the strong contacts between cellulose fibers to resist the inter-fiber slippage. As water-weakened hydrogen bonds between cellulose fibers were broken under stretching, intact hydrogen bonds served as contact points that pinned adjacent cellulose fibers supporting fiber reorientation and the formation of fiber ridges and valleys upon buckling. However, the presence of CNTs inhibited inter-fiber interactions, and resulted in sliding of cellulose fibers under stretching, rather than buckling. The sliding of cellulose fibers prohibited their orientation changes, thereby resulting in the lower degree of reorientation and thus, the lower auxeticity of CPC. This conclusion agreed with the larger auxeticity of CPC with the lower CNT contents as shown in FIG. 2D.

The Poisson's ratio describing the auxetic behavior was described with a global strain not a local strain as shown in equation (1) and (2). It was appropriate to use a global strain rather than a local strain because the stress concentration due to different Young's moduli and Poisson's ratios was the main factor for the large auxeticity. The large property difference of the wet and dry regions caused the stress concentration to increase the auxetic behavior. The stress concentration resulted in the necking of the wet region, and the subsequent larger buckling of the cellulose fibers. Hence, the Poisson's ratio was computed by the global strain not by the local strain.

### Sensing Performance and Applications of CPC Piezoresistive Sensor

FIGS. 4A-4F show characterizations of the sensing performance of a CPC piezoresistive sensor, in accordance with the present technology. FIG. 4A shows a normalized resistance response of CPC sensors with CNT wt % of 2.5, 5, 10, and 10 with V-shaped pattern under applied pressure from 0-500 kPa. On the vertical axis is the normalized resistance. The pressure in kPa is on the horizontal axis.

The CPC piezoresistive sensor showed high sensitivity with large dynamic range. The piezoresistive response was characterized for the pressure range of 0-500 kPa as shown in FIG. 4A. The sensitivity showed a descending trend as the applied pressure increased. The empirical correlation between the normalized resistance of 10 wt %-CPC and the applied pressure (P) was:

ΔRₙₒᵣₘ=9.0×10⁻¹⁶ p⁶−1.0×10⁻¹² p⁵+1.0×10⁻⁹ p⁴−3.0×10⁻⁷ p³+5.0×10⁻⁵ p³−0.0038 p+0.99 (4)

where ΔRₙₒᵣₘ is the normalized resistance of 10 wt %-CPC. The linearized sensitivities of 2.5, 5, and 10 wt %-CPC are shown in the pressure range of 0-50 kPa (FIG. 4b). In addition to the fracture induced by straight line water-printing, a V-shaped fracture could also be generated by water-printing. The sensitivity of V-shaped CPC with 10 wt % was compared to assess the effect of fracture area on sensitivity. As shown by the inset of FIG. 4b, the V-shaped CPC showed greater fracture area compared to the straightly fractured CPC. The sensitivities of 2.5, 5, 10 wt %-CPC, and V-shaped 10 wt %-CPC were (9.0±5.0)×10⁻³, (4.1±1.4)×10⁻³, (2.4±0.12)×10⁻³, and (3.3±0.25)×10⁻³ kPa⁻¹, respectively. The sensitivity of “V” shape-fractured sensors showed 1.38 times that of the straightly fractured sensors due to the 40% larger fracture area. The increase of the fracture area led to the sensitivity increase by the similar ratio, which proposed the facile methodology of manipulating the sensitivity of the piezoresistive sensor by producing a water-printed fracture pattern.

FIG. 4B shows the packaging of the pressure sensor, and the average sensitivity of CPC sensors with CNT wt % of 2.5, 5, 10, and 10 with V-shaped pattern under applied pressure from 0-50 kPa. On the vertical axis is the sensitivity in kPa⁻¹, and on the horizontal axis is the CNT w/w %. FIG. 4C is a fractured shape induced by straight line and V-shaped water-printing and shows the normalized resistance response of CNT-cellulose piezoresistive pressure sensor (thickness: 100 mm) to cyclic loads of 0-40 kPa. FIG. 4D is a closeup of normalized resistance response for 750-755 s. The CPC sensor is sealed with a polyethylene terephthalate (PET) film to avoid damaging the sensing element. Cyclic detection of small pressure of 50 Pa is shown. FIG. 4E is a graph of the sensor surface with and without the weight block. The resistance changes of the CPC sensor when detecting a small drop of water with applied pressure of 6 Pa and 13 Pa, respectively, are illustrated.

The repeatability of a CPC piezoresistive sensor was measured for 10 000 cycles at different compressive pressure as shown in FIG. 4C. For the cyclic pressure of 0-40 kPa, the sensor showed consistent resistance change. The sensing repeatability under smaller compressive loads was also demonstrated using a silicone block to apply a cyclic pressure of 50 Pa, which was successfully detected by the normalized resistance change of 0.02 as shown in FIG. 4D.

The CPC piezoresistive sensor exhibited an extremely low detection limit. FIG. 4E shows the detection of very small pressures 4310 Pa. The water drops of 10 and 100 mL were applied on a thin film placed above the fracture area of the sensor, with the contact area of 16 and 78 mm2, respectively.

The 10 mL water drop applied a pressure of only 6 Pa, resulting in a sensitivity of 3.3 kPa⁻¹. Opportunities exist to improve the detection limit further by designing CPC with greater fractured area. Note that the sensitivity could vary depending on the contact condition between an object and the sensor surface.

For example, the water contact on the sensor surface was more uniform than the silicone block, which resulted in the higher sensitivity.

The high sensitivity of a CPC piezoresistive sensor was attributed to the dramatic disconnections and reconnections of the molecular junctions in conjunction with the extreme auxeticity. Numerous electrical paths were established on as-prepared CPC, as demonstrated by the SEM that shows evenly dispersed CNTs on random network of cellulose fibers. The electrical paths broken by the fracture could be reconnected under the applied pressure, resulting in piezo-sensitivity. As the distance between CNTs became greater than the tunneling distance, the resistance increased according to the power law. The out-of-plane directional pressure reduced the distance between CNTs and induced the intensive recovery of the CNT connections. Hence, the piezoresistive sensors fabricated from the locally auxetic CPC demonstrated excellent sensitivity. Finally, FIG. 4F is a comparison of piezoresistive sensors for their sensitivity and dynamic range. In comparison to other random-network sensors, the disclosed sensors showed the outstanding performance in the sensitivity and dynamic range.

FIG. 5A is a CPC piezoelectric heartbeat sensor capable of measuring the rate of cardiovascular pulsations when wrapped around the wrist of an individual, in accordance with the present technology. The resistance variance of CPC pulse sensor when detecting wearer's pulse is shown.

FIG. 5B depicts a CPC piezoelectric sensor on a belt, in accordance with the present technology. The cyclic motion from thoracic or abdominal expansions and contractions during inhalation and exhalation was also detected by mounting a CPC piezoresistive sensor on a belt, as depicted. The belt tension was adjusted such that the respiration motion could generate adequate relative pressure. Illustrated in FIG. 5B is the normalized resistance of the smart belt during normal respiration.

FIG. 5C shows the resistance changes of a foot pressure sensor at three modes of motions, in accordance with the present technology. The three modes of motions are walking, running, and jumping. The CPC sensor is sealed with a polyethylene terephthalate (PET) film to avoid damaging the sensing element.

The pressure difference between a human body and a sensor could be captured by a CPC sensor. The sensor is insensitive to the belt strain because of the sensor covered with a PET film. This offered an inexpensive and reliable way of monitoring breathing patterns for applications in sports and neonatal care. In addition, a CPC sensor attached to an insole was able to monitor the gait movement based on foot pressure. Step count could be extracted from the piezoresistive signal. Walking, running, and jumping motions were clearly discriminated in the waveforms, as shown in FIG. 5C. The gait monitoring tests further confirmed that CPC sensors could sustain repeated stress at elevated pressure without hindering the sensing performance.

In summary, the controlled auxeticity of a random fibrous network comprising a cellulose paper composite grafted with carbon nanotubes was investigated in combination with an innovative water-printing method. The CPC was locally fractured with necking along a region due to the reduced CPC strength and stress concentrations. Due to the wetting—stretching method, the fracture process of CPC was reproducibly manipulated with six-time water-printing. It was discovered that the amplified auxetic behavior was a result of the buckling of wet CPC matrix during fracture. The effective Poisson's ratio of CPC achieved a value of −49.5. The auxetic behavior of CPC improved the piezoresistive sensitivity through the recovery of terminated electrical pathways upon applied pressure. A remarkable piezoresistive sensitivity of 3.3 kPa⁻¹ and a wide sensing range of 6-500 000 Pa were achieved. Tailoring auxeticity of a random matrix paper-based composite offers a new route to enhancing the piezoresistive sensitivity with the improved manufacturing reproducibility toward wearable applications, for instance, the gait and respiration detection.

FIGS. 6A-6C are graphs of the stress-strain relationship between 2.5%, 5%, and 10% CNT after no wetting, 2, 6, and 10 times of wetting in accordance with the present technology. On the vertical axis is stress in MPa, and on the horizontal axis is strain,

FIGS. 6D-6F are graphs of the wetting time in relation to the fracture strain of the CNT, the ultimate strength in MPa, and the wet strength retention, in accordance with the present technology.

### Example #2

The capacitive sensing mechanism of the fractured CPC composite was tested for humidity. The high aspect ratio of the cellulose fibers created by axial stretching enhance the electric field around the crack domain. Water molecules introduced on the surface of the crossing radial structure enlarge the capacitance change among the high aspect ratio electrodes, resulting in an extreme change of capacitance.

### Experimental Method

Materials included leached Kraft softwood pulp (SW), provided in a dried mat form from Port Townsend paper mill. Alkali lignin (AL, 99%), sodium dodecyl sulfate (SDS, 99%), and cationic polyacrylamide (CPAM, Percol 3035), which were obtained from Tokyo Chemical Industry Co., MP Biomedicals, and BASF, respectively. Hydroxyl-functionalized carbon nanotubes (CNT-OH), synthesized from catalytic chemical vapor deposition, were purchased from Cheap Tubes Inc. As per the manufacturer data, CNT-OH have lengths in the 10-20 μm range with the mean diameter of 50 nm, with an average of of OH groups. All chemicals were used as received without any additional treatment.

CNT-cellulose composite papers were prepared following a modified TAPPI T-205 standard method, as previously reported elsewhere. Briefly, hand-sheets were formed by a filtration method using a hand-sheet molder (Essex International Inc. Custom Machinery) and pressed and dried according to TAPPI T-205 standards. Prior to sheet formation, CNT-OH were dispersed in a binary mixture of AL and SDS (90:10 wt) using a double acoustic irradiation system, to promote individual dispersion in solution and achieve a uniform distribution of charge transport routes throughout the final composite. Aqueous dispersion of CPAM were first added to pulp fiber solutions (0.3% consistency) and combined on a hot plate at 50° C. for 30 minutes. The as-dispersed CNT-OH solutions were then added to the pulp mixture and kept under constant agitation for 30 minutes. The combined CNT-OH and pulp suspensions were then filtered, pressed, and dried to form hand-sheets. The proportion of cellulose fibers, CNTs, CPAM, AL, and SDS were adjusted to achieve a total mass of 1.2 g OD (60 g m⁻²). For comparison purposes, hand-sheets were also prepared without any CNT-OH, just using a pulp/CPAM/AL/SDS blend and denoted as “control” samples. All hand-sheets were kept for 48 hours at room temperature conditions (23° C.) and 50% relative humidity prior to testing. All hand-sheets had a mean thickness of 88.4±3.1 μm.

CPC capacitive sensors were fabricated by controlled water-printing and axial stretching (Reference). Silver paste (MG Chemicals, USA) was applied to both ends of the CPC strip and cured at 70° C. on a hot plate to make electrodes. Using a 0.7 mm-diameter capillary pen, water was printed without a physical contact to CPC.

To produce auxetic behavior, a tensile testing stage was constructed with a uniaxial actuator. The tension was applied with a constant speed of 37.5 micron/s. To study the effect of humidity to auxeticity, humid air was continuously supplied to a CPC specimen through a 12 mm-diameter nozzle in a tensile test. Force and resistance were recorded by a load cell (DYMH-103, CALT, China) and a multimeter (Fluke Corp., USA), respectively. The stress was calculated by

$$σ = \frac{F}{D \times T} ,$$

where F was the force measured by the load cell, D was the initial width of a specimen, and T was the initial thickness (i.e. 100 μm) of the specimen measured by a digital gage (PK-0505, Mitutoyo, Japan). The axial strain was

$$ε = \frac{l - {l}_{0}}{{l}_{0}} .$$

For comparison of auxeticity and capacitive change, CPC specimen without water-printing were also tested.

Auxeticity of a CPC sample is related to the compression and buckling of a specimen in the width direction. To investigate the width effect on auxeticity, the specimen widths of 1, 3, 5, 7, and 10 mm were prepared. The CPC auxetic behavior was studied by measuring the thickness changes. FIG. 7A is a test setup to investigate the auxetic behavior of the CPC, in accordance with the present technology.

In the testing stage as shown in FIG. 7A, a microscope was focused on the region of the specimen from the top- and side views of the stretching. The thickness change of a specimen during the water-printing and stretching was measured. The effective Poisson's ratio of CPC with the CNT concentrations of 10 wt % were computed by the following equations:

$${v}_{eff} = - \frac{( {z}_{i} - {z}_{0} ) / {z}_{0}}{( {l}_{i} - {l}_{0} ) / {l}_{0}} \qquad ( 5 )$$

where lᵢ and zᵢ denoted the specimen length and thickness values at the given strain level, and lᵢ−1 and zᵢ−1 denoted their values at the previous level. l₀ and z₀ denoted the original specimen length and thickness. For both specimens with and without the water-printing. v_{eff} were computed at strain ranging from 0˜0.36. The v_{eff} of non-water printed paper and CPCs were computed at the fracture strains.

Scanning electron microscopy (SEM, XL830, FEI Company, Hillsboro, OR, USA) was used to study the CPC surface morphology in-plane and fracture length. To ascertain the fracture length and morphology, the CPC was mounted to a flat aluminum stage using double-sided carbon tape and imaged using a 5 kV accelerating voltage with a 5 mm working distance.

The resistive and capacitive changes of a CPC sensor were studied for the CPC specimen stretched with various strains of 0.10, 0.12, 0.15, 0.18, and 0.24. At each strain, the specimen was placed at 30%-RH for the first 20 seconds, followed by the application of 100%-RH air. The intensive humid air was supplied directly to the sensor for 50 seconds. The outlet nozzle of humid air was located at 10 mm above the top surface of specimen. Subsequently, humid air was removed to leave the sensor at RH 30% for 110 seconds. Therefore, the total time of experiment for each applied strain was 180 seconds. The resistance and capacitance values were measured by a Fluke meter and a capacitance meter (GLK 3000), respectively. Meanwhile, a commercial humidity sensor was located next to a CPC specimen in order to measure the humidity change.

A CPC specimen with 0.24-strain was placed in a 5L-humidity chamber. The humidity was controlled by a humidifier and a vacuum pump. The humidity was controlled for 10 cycles between RH 37% and 100%. In the chamber, a reference humidity sensor was used to measure RH at the rate of 1 sample/s. The capacitance values were measured using a capacitance meter (GLK 3000).

To investigate the humidity sensing mechanism of a fractured CPC sensor, three differently treated CPC sensors and one aluminum sensor were prepared for a cyclic humidity testing. Among three kinds of CPC sensors, three were a fractured CPC sensor as prepared by 0.24-strain, a fractured sensor coated with polyacrylic acid (PAA), and a fractured CPC sensor laminated with a 20 μm-thick polyester film. The other was a CPC sensor trimmed with scissors without fracture. An aluminum sensor was prepared by trimming a 100 μm-thick aluminum foil. All the surface area of one electrode was 5×5 mm². The PAA-coated CPC was prepared to check if the swelling ability of cellulose fibers could enhance the capacitive sensitivity. 1% PAA-solution was deposited into a CPC sensor and cured for one hour on a hot plate. After curing, the sensor was fractured by introducing 0.24-strain. A fractured CPC sensor laminated with a polyester film was used to test capacitive sensitivity. In comparison to a fractured CPC sensor without lamination, the response of a laminated sensor could give information about the capacitive sensing mechanism if the sensitivity was resulted from the cantilever-shaped electrodes or the CNT surface change. A scissor-trimmed CPC sensor was used to study a humidity sensitivity without cantilever-shaped electrodes. Scissor-trimmed aluminum electrodes were fabricated in the same way as scissor-trimmed CPC electrodes. A scissor-trimmed aluminum capacitance was prepared to study the CNT surface change in comparison to aluminum surface.

A cyclic humidity testing was conducted by supplying humid air into a chamber of 3.8 L. The humidity was controlled between RH 37% to 95%. The humidity change was repeated for four cycles to study the reproducibility. The capacitance change was measured by GLK 3000. A reference humidity sensor was used as a control.

### CPC Stretching Characterization

The CPC sensors were fractured under a condition using the setup as shown in FIG. 7A. To study the capacitive and resistive changes in coupling with the auxeticity, three CPC sensors were stretched in the same loading condition with and without water printing. Optical microscopes were placed to observe the top- and side views of the fracture process. From the top-view images, the CPC samples without and with water printing were clearly differentiated. The crack of the sensor was propagated along the water line, perpendicular to the stretching direction. The crack of the CPC without water printing was propagated at a 45-degree angle to the stretching direction due to the shear failure as shown in FIG. 7B.

FIG. 7B is a fractured CPC with and without water printing, in accordance with the present technology. Then, the thickness change was recorded by the side-view microscope. The thickness change was used to calculate an effective Poisson' ratio through equation (5).

### Capacitance Characterization for a Humid Test of CPC Sensors

The strength for a water printed CPC was lower than that of CPC without water printing. FIG. 7C is a graph of the stress-strain relationship for the CPC with and without water printing, in accordance with the present technology. Resistance change is described on the second y-axis. The resistance increased by a power law due to rapid increase of percolation.

The capacitive response of the CPC sensors with and without water printing were characterized under an RH-100% condition. The nozzle connected to a humidifier was applied directly on the top sample surface in stretching. Capacitance change was measured in terms of the applied axial strain in FIG. 7D.

FIG. 7D is the capacitance change for CPC with and without water printing, in accordance with the present technology. The capacitance of both samples with and without water printing started with negative values because the produced capacitance was in parallel with the electrical resistance. The negative capacitance means the leakage of electric current through resistive connection of CPC. As a CPC specimen started to fracture, the negative capacitance value increased. Note that the dip in the negative capacitance was the characteristic of a capacitance meter circuit. As the strain crossed 0.1, the negative capacitance of CPC with water-printing became positive while the CPC without water-printing stayed at a negative value. Interestingly, the CPC with water-printing went the maximum value of 103.3 pF and converged to zero as the distance between two fractured CPC increased. The two capacitance curves met at 0.24-strain where the samples were completely terminated electrically and mechanically.

FIG. 8A-8C are SEM images at 0.12, 0.15, and 0.18 strain, in accordance with the present technology. FIG. 8D is a graph of the normalized thickness change according to axial strain for CPC with and without water printing, in accordance with the present technology.

FIG. 8A-8C show the SEM images of the cross section according to 0.12, 0.15, and 0.18 strain, respectively. In comparison to the thickness of the specimen without water-printing, the thickness increase of that with water printing was greater, as shown in FIG. 8D. The thickness increase reached the maximum value when the applied strain was 0.24. As the specimen was completely fractured, the thickness reduced slightly with complete release of the tensional force.

FIG. 8E is a graph showing the Poisson's ratio according to specimen widths, in accordance with the present technology. As the width increased, the Poisson's ratio increased. FIG. 8F is a graph showing the maximum capacitance according to sample widths, in accordance with the present technology. As the width increased further, the capacitance increase was rapid due to the larger auxeticity, and thus the greater capacitance. However, the capacitance increase was saturated due to the periodic buckling at a larger width.

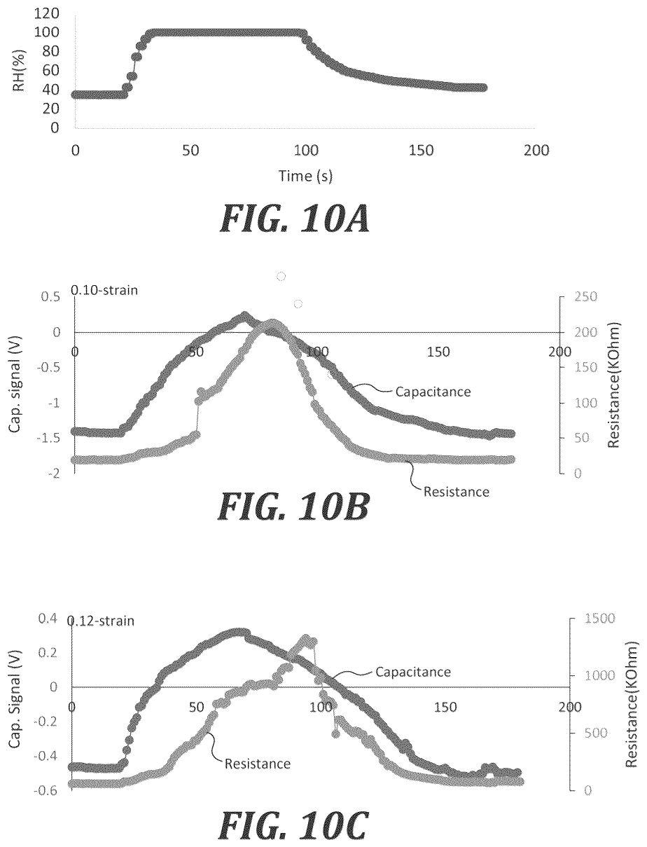

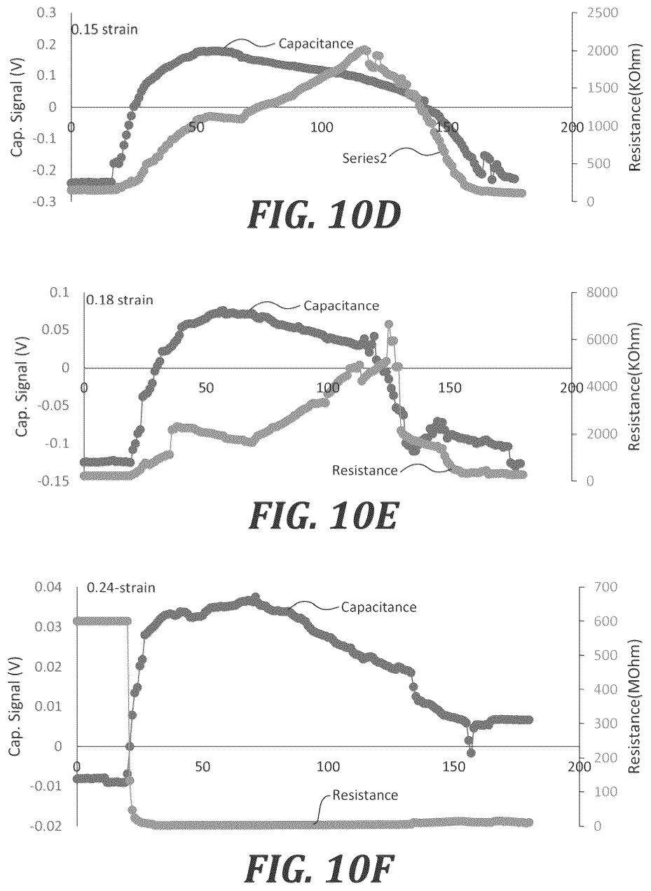

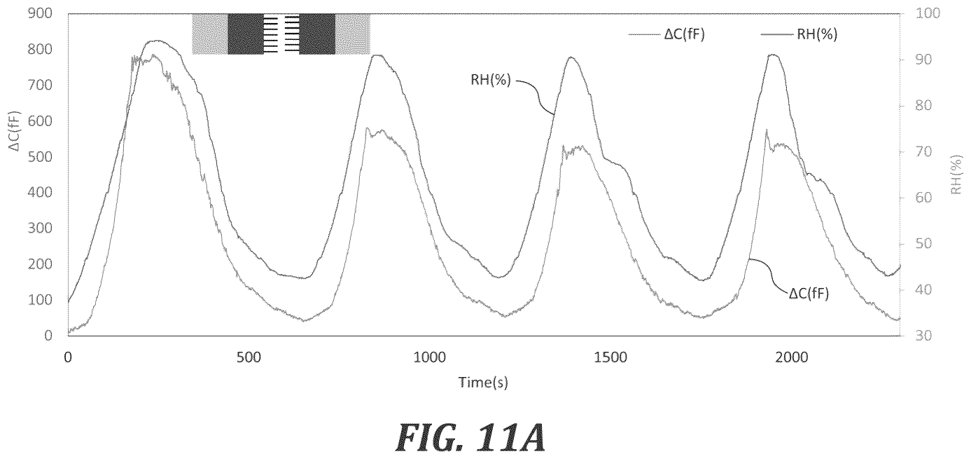

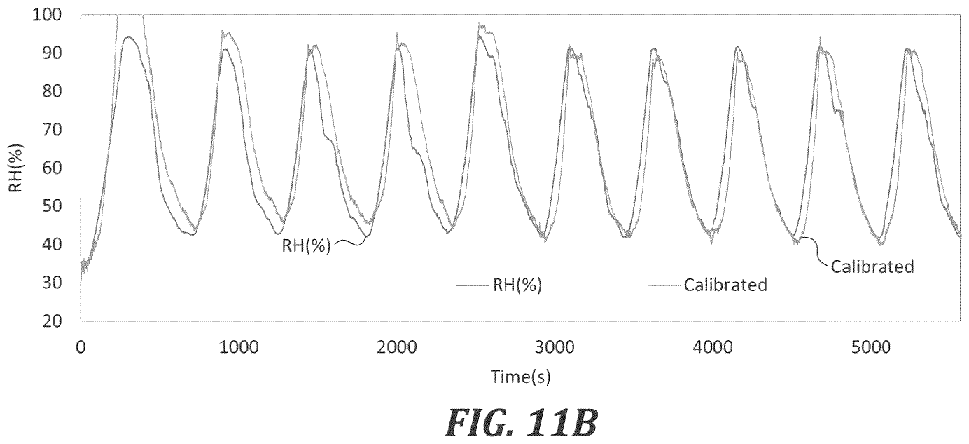

FIG. 9A is the stress distribution on a 1 mm width CPC strip resulting from the compression, in accordance with the present technology. FIG. 9B is the stress distribution on a 3 mm width CPC strip, in accordance with the present technology. FIG. 9C is the compressive stress built across the width, in accordance with the present technology. At a width greater than 3 mm, the buckling occurs.

The auxeticity was related to the width of a CPC specimen, which was validated by COMSOL simulation. The 1 mm-displacement was applied on the right end at the longitudinal direction to simulate the fixed-strain tensile deformation. The other y- and z-directions were fixed at both ends. The left end of the specimen was fixed. All other boundaries were treated as free ends, and a tetrahedral mesh was used. Because of the positive x-y Poisson's ratio, a compression was generated across the central region along the y direction, as seen in FIGS. 9A-9C. The compression force was used to estimate the compression force at the wet region.

The averaged compressive stress is then compared with that of the critical y directional buckling force of the central region under the pin-joint conditions, which was calculated as:

$${P}_{cr} = \frac{{π}^{2} EI}{{L}^{2}}$$

where the moment of inertia is evaluated across x-axis and L is the width of the CPC strip. FIG. 9D is a graph showing that at 1 mm width, the averaged engineering stress cannot buckle the central region, in accordance with the present technology. The numerical results indicated that the compression stress could cause buckling of a specimen wider than 2 mm. As the width increased over 3 mm, the CPC specimen could buckle due to the increased slenderness ratio. The buckling increased the Poisson's ratio and auxeticity. When the width was greater than 3 mm, the CPC specimen could be buckled with a periodicity, which explained the reduced slope of a capacitance

### Resistance and Capacitance Characterization of Humidity Test

To investigate the resistance and capacitance change to the humidity for various axial strain, the CPC samples with the applied strain of 0.1, 0.12. 0.15, 0.18, and 0.24 were placed in a chamber of RH-30% (25° C.). The 0.1-strain was a starting value because positive capacitance value initiated with the fracture of CPC specimen. Subsequently, a nozzle with a humid air was directly applied for 50 seconds and removed as measured by a reference humidity sensor in FIG. 4a. During the humidity experiment, both resistance and capacitance were measured by Fluke meter and GLK 3000, respectively.

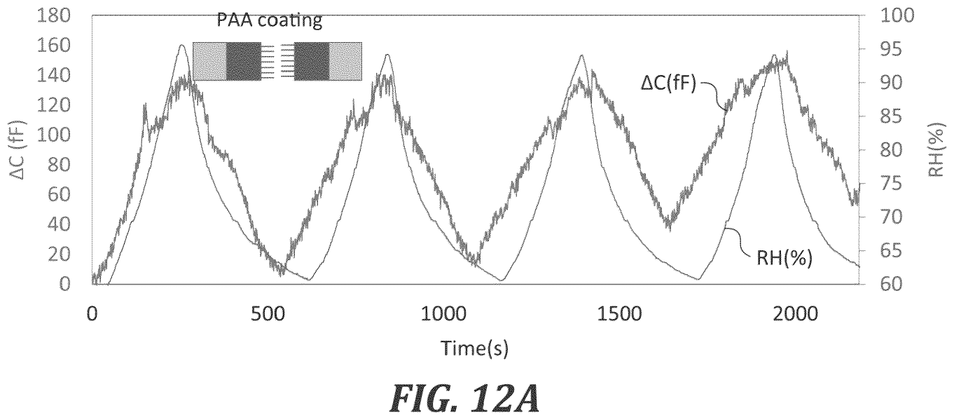

FIGS. 10A-10F are graphs showing the resistance and capacitance change of the specimen of 0.10, 0.12, 0.15, 0.18 and 0.24-strain for the humidity change, in accordance with the present technology. The equivalent circuit of the CPC samples was the parallel connection of resistance and capacitance. For the CPC with 0.10, 0.12, and 0.15, the resistance gradually increased as the humid air was supplied and then reached to a plateau at 50 seconds. However, when the humid air was removed at 70 s, the resistance increased again. As the applied strain was greater, the duration time of the resistance value increased. When the sensor was exposed to humid air, the resistance increase was originated from the resistance change of MWCNTs on the fibers due to water molecules. The CNT adsorbing water molecules led to reduction of hole concentration of CNTs. As RH increased further, the resistance change was then dominated by losing CNT electrical junctions because of fiber swelling. This phenomenon was also found and reported in 2013. In our experiment, the resistance change was even higher because the auxeticity at fracture produced a larger volume for water adsorption than the intact CPC with high percolation. The hydro-expansion of cellulose fibers prevented electrical interactions among CNTs, therefore increased the composite resistance. Although humid air was removed at 70 s, water molecules were left on the surface of the composite due to the intense humidity. With the larger expansion, we observed a longer time of the second increase of the resistance because of larger surface area for water absorption. However, for the CPC with 0.24-strain, the sensor was just entirely terminated with infinite resistance. For plotting purpose, instead of infinity, the beginning point of resistance was set as 500 MOhm, which was the maximum measurable resistance of the Fluke meter. On the contrary, the resistance value decreased from infinity to several MΩ. Since all the fibers at the fracture domain were untangled, the sensor behaved as a pure capacitor. However, the intensive humid air would form water junction among the conductive fibers, which made electrical connection.

For the same CPC, the capacitance did not show the second leap. After removing humid air, the rising trend was changed to a descending trend. The capacitance values for all the samples changed in the similar way but with different magnitude. The capacitance began to rise at the point of humid air and declined upon the removal of humid air. The highest capacitive sensitivity of CPC sensor was right after fracture. The magnitude of the capacitance change due to the humidity was descending with the larger axial strain.

### Calibration of a Capacitive Humidity Sensor made of Fractured CPC

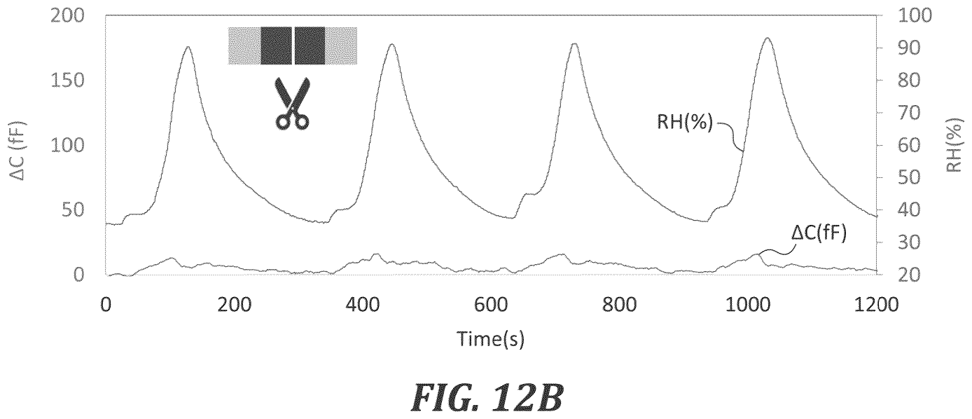

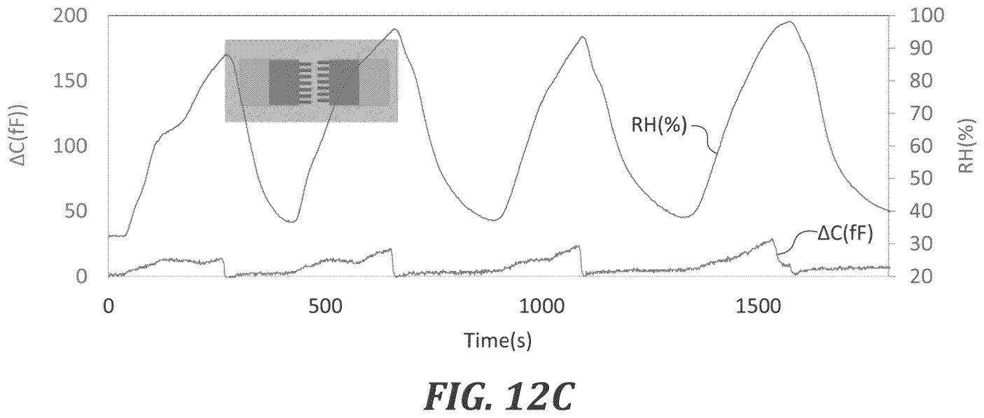

FIG. 11A is a graph of the capacitance change of a fractured CPC sensor according to humidity change, in accordance with the present technology. FIG. 11A illustrates the capacitance change for a CPC humid sensor inside a chamber for 10 cycles humidity changing 35˜95%-RH. Except the first two cycles, the measured capacitance values were stable and reproducible. Using the data from the third cycle, the empirical correlation between the capacitance value and the relative humidity was obtained:

$$R H = 84 - ( 52.75 - \frac{\sqrt{80000 - 42255 x )}}{4} ) \qquad ( 6 )$$

where x is the capacitance value.

FIG. 11B is a graph of the comparison of the fractured CPC-humidity response to a commercial sensor, in accordance with the present technology. FIG. 11B illustrates the comparison between the calibrated RH data of a CPC sensor using equation (6) and the RH data measured by the reference humidity sensor, which showed a good agreement. For the measurement obtained after the initial cycle, the response became repeatable and stable.

### Humidity Sensing Mechanism

To investigate the capacitive sensing mechanism of humidity, CPC sensors coated with PAA, a polyester film, and trimmed with a scissor. FIGS. 12A-12 are graphs of the capacitive changes of PAA-coated CPC, trimmed-CPC, plastic-film-coated CPC, and trimmed aluminum sensors for cyclic humidity change, in accordance with the present technology. A metallic capacitive sensor was also prepared by trimming aluminum foil with the same dimensions.

FIG. 12A is a graph of the capacitance change of a fractured CPC coated with PAA. The PAA-coated CPC sensor showed the multistage swelling effect. When contacting with water vapor, both PAA and fibers could swell with hydro-expansion, which showed the phase shift. The results clearly showed that the capacitive change of fractured CPC was not originated from the resistive change but the capacitive change on the surface of the fractured fibers.

A scissor-cut CPC sensor without fracture showed a negligible sensitivity to humidity as shown in FIG. 12B. The fractured CPC coated with a PET film showed the change of 20 fF because the adsorption of water molecules was blocked by a plastic film as shown in FIG. 12C. The sensitivity was higher than those of trimmed CPC sensors due to the higher electric field strength. FIG. 12D shows a metallic capacitive sensor insensitive to humidity change, which was similar to a trimmed CPC sensor. Since the permittivity change was negligible in humid air, the capacitive change was negligible.

The high capacitive sensitivity of the fractured CPC composite to the humidity was coupled with the high aspect ratio cantilever structure generated by stretching and the permittivity change of the adsorbed water molecules on the surface of cantilever fibers. In fracture, the randomly oriented fiber networks became straight. When these fibers were exposed to water vapor, the water molecules could adsorb on the surface area where a high electric field was produced to form capacitance. When the strain increased, the fewer fibers could be in contact, therefore, lower sensitivity.

### Application for Sweat Sensing

FIG. 13A shows a chamber to measure humidity change on a hand, in accordance with the present technology. FIG. 13B is a graph of the capacitance change measured on palm, in accordance with the present technology. The humidity change of a capacitive CPC sensor shows a good agreement with that of a resistive commercial sensor.

A fractured CPC sensor with 0.24 strain could be used to evaluate the water evaporation of human's skin. To test a CPC sensor, a small chamber with an evaporation hole to contain a commercial humidity sensor and a CPC sensor was constructed, as shown in FIG. 13A. When a CPC sensor was placed in the center of palm, the sweat evaporation was detected from human hand. The data obtained from a CPC sensor were measured using a capacitance-to-digital chip (FDC1004). The RH reached 85% when the chamber was placed on the palm. The RH decreased to RH-55% when the sensor was removed from the palm. The calibrated humidity data for CPC were compared to those of a commercial sensor FIG. 13B. The calibrated data showed a good agreement with the reference commercial sensor.

For humidity sensing, resistive and capacitive sensors are available. Between two electrodes, a humidity absorption pad is applied to change a resistance or the permittivity to capacitance. Using CNTs, a humidity sensor was investigated for a resistive sensor due to the absorption of water molecules changes. The resistance change of CPC coated with PAA was also sensitive to humidity due to a swelling effect. The fractured CPC capacitive sensor was novel in that the capacitive change to humidity was significant without an absorption medium. This capacitive measurement was unusual in that the air permittivity change due to humidity was negligible. The high electric field contributed to the sensitive measurement to humidity. According to our numerical simulation, the electric field could increase to 107 V/m considering the gap size. When the fractured fibers coated with CNTs were blocked with a polyester film, the humidity change was still detectable but a reduced sensitivity. The experimental results showed that the major capacitive response was resulted from the change of CNT surface on cellulose fibers in coupling with a high electric field.

Paper made of cellulose, the most abundant natural polymer extracted from woody biomass, has the benefits of being low-cost, lightweight, and having a large surface area. The nonwoven structure of cellulose fibers provide the random networks with auxeticity. This auxetic material shows piezo-resistivity when assembled with sensing elements. However, the low auxeticity of cellulose fiber networks barely contributes to the sensitivity. The constraints of inter-fiber junctions hampered large deformations of cellulose network and disconnections of molecular junctions. The fracture of CPC reorganized the cellulose networks provides an insight for the in-plane electromechanical coupling of the random networks under structural reorganization. However, the inconsistent and dispersive fracture shows unpredictable sensitivity, and the contribution of the auxetic behavior was not clear.

The large capacitance change was resulted from the auxetic behavior caused by the buckling of a specimen. The sensitivity of the sensor due to RH cycles was observed in the controlled humidity chamber and calibrated with a reference humidity sensor. According to the test results, a capacitance reached a maximum value where the fracture of the CPC composite was just occurred. The magnitude of Poisson's ratio was also the maximum at the point. An empirical equation for the capacitance value and RH curve was obtained by calibration with a reference humidity sensor. The calibrated fracture CPC humid sensor could also be used for sweat measurement in our hand. Hence, the fractured CPC capacitive sensor is capable of sensing humidity without absorption medium because the auxetically-produced cantilever shaped electrodes form very sensitive capacitive junctions. The capacitive sensing platform may facilitate a wearable sensor detecting humidity and moisture change.

1. A sensor, comprising:
a composite substrate comprising a template material, wherein the template material comprises:
a plurality of insulating fibers; and
a plurality of carbon nanotubes bonded to at least a portion of the insulating fibers forming a nanotube coating on the insulating fibers;
wherein the composite substrate exhibits a tensional fracture induced by a unidirectional tensile force to the composite substrate, wherein the plurality of insulating fibers align along the tensile force and expand in an out-of-plane direction at or near the site of the fracture; and
a first electrode on one side of the fracture.

2. The sensor of claim 1, wherein the insulating fibers are compressed in the width direction and expand out of plane with buckling to align fibers along the tensional direction.

3. The sensor of claim 1, wherein the composite substrate is wet with a liquid at or near the site of the fracture when the unidirectional tensile force is applied to the composite substrate.

4. The sensor of claim 3, wherein the liquid is printed onto the composite substrate to form a liquid printed region.

5. The sensor of claim 4, wherein the liquid printed region is a V, a W, a circular shape, or a random shape.

6. The sensor of claim 3, wherein the liquid aids in initiating and designing a cracking pattern in the composite substrate.

7. The sensor of claim 4, wherein the fibers fracture at or near the liquid printed region under a high relative humidity environment having a humidity between about 80% to 100% humidity.

8. The sensor of claim 7, wherein the liquid printing is repeated under low humidity environment having a humidity between 0 to about 80% humidity in order to make the composite fully wet.

9. The sensor of claim 3, wherein the surface area of the composite substrate has an increased surface area at or near the site of the fracture.

10. The sensor of claim 3, wherein the composite substrate has a plurality of high aspect ratio cantilevered structures at or near the site of the fracture.

11. The sensor of claim 10, wherein the plurality of cantilevered structures is aligned along the tensional direction.

12. The sensor of claim 1, wherein the sensor is an in-plane strain sensor, an out-of-plane piezo-resistive sensor, or a capacitive sensor.

13. The sensor of claim 1, wherein the sensor is a heartbeat sensor, a gripping motion sensor, a breathing sensor, a nasal air flow sensor, a finger movement sensor, a proximity sensor, or a human-machine interface.

14. The sensor of claim 1, wherein the sensor is a humidity sensor configured to measure humidity and environmental gas composition change.

15. The sensor of claim 1, wherein the sensor is a bistable resistance-capacitance component that is controlled by humidity.

16. A method of making a sensor, comprising:
acquiring a composite substrate comprising a template material, wherein the template material includes a plurality of insulating fibers, and a plurality of carbon nanotubes bonded to the insulating fibers forming a nanotube coating on the insulating fibers; and
applying a unidirectional tensile force to the composite substrate, creating a tensional fracture, wherein the plurality of insulating fibers align along the tensile force and bulge with out-of-plane direction at or near the site of a fracture, and wherein the composite substrate includes a first electrode on one side of the fracture.

17. The method of claim 16, the method further comprising:
printing a liquid on the composite substrate in a liquid printed region prior to applying a unidirectional tensile force; and fracturing the insulating fibers at or near the liquid printed region.

18. The method of claim 17, the method further comprising applying a unidirectional tensile force under a high relative humidity environment having a humidity between about 80% to 100% humidity.

19. The method of claim 18, wherein the liquid printing is repeated under low humidity environment having a humidity between 0 to about 80% humidity in order to make the composite fully wet.

20. A sensor manufactured by the method of claim 16.

21. The sensor of claim 1, further comprising a second electrode on the opposite side of the fracture, such that an electrical signal configured to be applied between the first electrode and the second electrode passes through the site of the fracture.

22. The method of claim 16, wherein the composite substrate further includes a second electrode on the opposite side of the fracture, such that an electrical signal configured to be applied between the first electrode and the second electrode passes through at the site of the fracture.
